(19) United States
(12) Patent Application Publication
(10) Pub. No.: US 2026/0132151 A1
(43) Pub. Date: May 14, 2026

(54) NOVEL N,N-DIMETHYLTRYPTAMINE (DMT) DERIVATIVES AND USES THEREOF

(71) Applicant: MiHKAL GmbH, Allschwil (CH)

(72) Inventor: Markus BUCHNER, Ottobrunn (DE)

(73) Assignee: MiHKAL GmbH, Allschwil (CH)

(21) Appl. No.: 19/135,759

(22) PCT Filed: Dec. 6, 2023
(86) PCT No.: PCT/EP2023/084586

§ 371 Date: Jun. 4, 2025

(30) Foreign Application Priority Data
Dec. 6, 2022 (EP) 22211625.3

(51) Int. Cl. C07F 5/02; A61K 31/405; A61K 31/69; C07D 209/16

(52) U.S. Cl. CPC C07F 5/025; A61K 31/405; A61K 31/69; C07D 209/16

(57) ABSTRACT

The present invention relates to compounds of formula (I), and pharmaceutically acceptable salts thereof. The present invention further relates to the compounds of formula (I) for use as a medicament and to a pharmaceutical composition comprising said compounds. The compounds provided herein can act as prodrugs that, upon administration, release N,N-dimethyltryptamine (DMT) or a derivative thereof and exhibit improved pharmacokinetic properties, which renders these compounds highly advantageous for use in therapy.

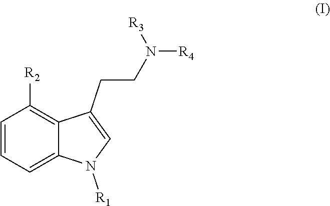

### FIELD OF THE INVENTION

The present invention relates to compounds of formula (I) and pharmaceutically acceptable salts thereof. The present invention further relates to the compounds of formula (I) for the use as a medicament and to a pharmaceutical composition comprising said compounds. The compounds provided herein can act as prodrugs that, upon administration, release N,N-dimethyltryptamine (DMT) or a derivative thereof and exhibit improved pharmacokinetic properties, which renders these compounds highly advantageous for use in therapy.

### BACKGROUND OF THE INVENTION

N,N-Dimethyltryptamine (DMT), an indole alkaloid widely found in nature, is a promising psychoactive compound for the treatment of anxiety, treatment-resistant depression and alcohol/drug dependence.

Its simple molecular structure, low molecular weight and hydrophobic nature allow DMT to cross the blood-brain-barrier (BBB). Furthermore, several studies have shown that DMT does not cause tolerance reactions in the human body. However, DMT is not orally available when administered alone. The low bioavailability can be explained by its rapid deamination by intestinal and hepatic monoamine oxidase-A (MAO-A). Therefore, oral consumption of DMT requires concomitant consumption of (natural) MAO-inhibitors, such as beta-carboline harmala alkaloids found in traditional ayahuasca mixtures. However, ayahuasca may be associated with adverse effects such as increased blood pressure, nausea and hyperthermia.

Modifications of DMT resulting in improved bioavailability and making simultaneous use of MAO-inhibitors redundant are of increasing pharmaceutical interest. However, tryptamines such as 5-methoxy-N,N-dimethyltryptamine (5-MeO-DMT) exhibit even higher affinities towards the serotonin receptor family and/or other receptor systems. Due to these altered characteristics, these developments have so far not resulted in a therapeutic product.

Thus, there is still an unmet need for DMT derivatives with improved therapeutic properties.

### BRIEF SUMMARY OF THE INVENTION

The present invention addresses this need by providing novel DMT derivatives having improved bioavailability compared to DMT. The compounds described herein may act as a prodrug of DMT and can readily liberate DMT under physiological conditions, as demonstrated in the in vitro experiments described in the examples section. Thus, the present invention provides compounds that act as prodrugs of DMT and exhibit improved pharmacokinetic properties (as compared to DMT), which renders these compounds highly advantageous for therapeutic use.

The compounds of the present invention are suitable for use as a medicament, e.g. for use in treating one or more diseases selected from anxiety disorder, attention deficit hyperactivity disorder (ADHD), depression, cluster headache, diminished drive, burn-out, bore-out, migraine, Parkinson's disease, pulmonary hypertension, schizophrenia, an eating disorder, nausea, vomiting, alcohol/drug dependency, or post-traumatic stress disorder (PTSD).

In a first aspect, the present invention relates to a compound according to the general formula (I), or a pharmaceutically acceptable salt thereof

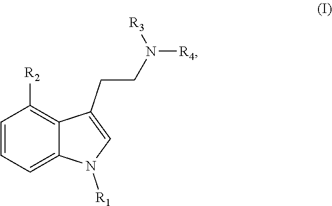

wherein:
R₁ is selected from the group consisting of hydrogen,

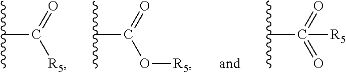

wherein R₅ is selected from C₁₋₁₂ alkyl (linear or branched), C₂₋₁₂ alkenyl (linear or branched), C₂₋₁₂ alkynyl (linear or branched), —CH₂—O—(CH₂CH₂O)ₘ—R₆, —O—(CH₂CH₂O)ₙ—R₆, —NH(CH₂CH₂O)ₚ—R₆, phenyl, benzyl, phenethyl, pyridyl, cycloalkyl, (cycloalkyl)alkyl, cycloalkenyl, (cycloalkenyl)alkyl, 5- or 6-membered heterocyclyl, (5- or 6-membered heterocyclyl)alkyl, and 9-fluorenylmethyl, wherein each one of the aforementioned groups is optionally substituted;
m, n and p are each independently an integer from 1 to 5;
R₆ is hydrogen or is selected from C₁₋₆ alkyl (linear or branched), aryl, heteroaryl, cycloalkyl, and heterocyclyl, wherein each one of the aforementioned groups is optionally substituted;
R₂ is selected from hydrogen and

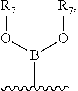

wherein each R₇ is independently selected from hydrogen and R₈, or wherein the two groups R₇, together with the —O—B—O— moiety that they are attached to, form a heterocyclyl which is optionally substituted;
wherein each R₈ is independently selected from C₁₋₁₂ alkyl (linear or branched), C₂₋₁₂ alkenyl (linear or branched), C₂₋₁₂ alkynyl (linear or branched), —CH₂—O—(CH₂CH₂O)ₘ—R₉, —O—(CH₂CH₂O)ₙ—R₉, —NH(CH₂CH₂O)ₚ—R₉, phenyl, benzyl, phenethyl, pyridyl, cycloalkyl, (cycloalkyl)alkyl, cycloalkenyl, (cycloalkenyl)alkyl, 5- or 6-membered heterocyclyl, (5- or 6-membered heterocyclyl)alkyl, and 9-fluorenylmethyl, wherein each one of the aforementioned groups is optionally substituted;
wherein m, n and p are as defined above;
wherein R₉ is hydrogen or is selected from C₁₋₆ alkyl, aryl, heteroaryl, cycloalkyl, and heterocyclyl, wherein each one of the aforementioned groups is optionally substituted;
R₃ is selected from hydrogen, methyl and ethyl;
R₄ is selected from hydrogen, methyl and ethyl; and
wherein at least one of R₁ and R₂ is not hydrogen.

In one embodiment, R₂ is

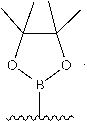

In another embodiment, R₁ is

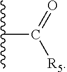

In another embodiment, R₁ is

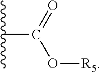

In another embodiment, R₁ is selected from

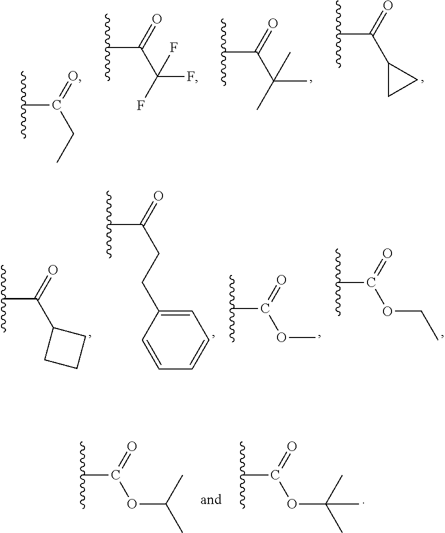

In yet another embodiment, the compound of formula (I) is selected from the group consisting of:
N,N-dimethyl-2-(4-(4,4,5,5-tetramethyl-1,3,2-dioxaborolan-2-yl)-1H-indol-3-yl)ethan-1-amine,
1-(3-(2-(dimethylamino)ethyl)-1H-indol-1-yl)propan-1-one,
1-(3-(2-(dimethylamino)ethyl)-1H-indol-1-yl)-2,2,2-trifluoroethan-1-one,
1-(3-(2-(dimethylamino)ethyl)-1H-indol-1-yl)-2,2-dimethylpropan-1-one,
cyclopropyl(3-(2-(dimethylamino)ethyl)-1H-indol-1-yl)methanone,
cyclobutyl(3-(2-(dimethylamino)ethyl)-1H-indol-1-yl)methanone,
1-(3-(2-(dimethylamino)ethyl)-1H-indol-1-yl)-3-phenylpropan-1-one,
methyl 3-(2-(dimethylamino)ethyl)-1H-indole-1-carboxylate,
ethyl 3-(2-(dimethylamino)ethyl)-1H-indole-1-carboxylate,
isopropyl 3-(2-(dimethylamino)ethyl)-1H-indole-1-carboxylate,
tert-butyl 3-(2-(dimethylamino)ethyl)-1H-indole-1-carboxylate,
2-(1-(cyclopropylsulfonyl)-1H-indol-3-yl)-N,N-dimethylethan-1-amine,
2-(1-(ethylsulfonyl)-1H-indol-3-yl)-N,N-dimethylethan-1-amine, and
2-(1-(methylsulfonyl)-1H-indol-3-yl)-N,N-dimethylethan-1-amine;
or a pharmaceutically acceptable salt thereof.

Preferably, the compound of formula (I) is selected from:
N,N-dimethyl-2-(4-(4,4,5,5-tetramethyl-1,3,2-dioxaborolan-2-yl)-1H-indol-3-yl)ethan-1-amine,
1-(3-(2-(dimethylamino)ethyl)-1H-indol-1-yl)propan-1-one,
1-(3-(2-(dimethylamino)ethyl)-1H-indol-1-yl)-2,2,2-trifluoroethan-1-one,
1-(3-(2-(dimethylamino)ethyl)-1H-indol-1-yl)-2,2-dimethylpropan-1-one,
cyclopropyl(3-(2-(dimethylamino)ethyl)-1H-indol-1-yl)methanone,
cyclobutyl(3-(2-(dimethylamino)ethyl)-1H-indol-1-yl)methanone,
1-(3-(2-(dimethylamino)ethyl)-1H-indol-1-yl)-3-phenylpropan-1-one,
methyl 3-(2-(dimethylamino)ethyl)-1H-indole-1-carboxylate,
ethyl 3-(2-(dimethylamino)ethyl)-1H-indole-1-carboxylate,
isopropyl 3-(2-(dimethylamino)ethyl)-1H-indole-1-carboxylate, and
tert-butyl 3-(2-(dimethylamino)ethyl)-1H-indole-1-carboxylate.

In a further aspect, the present invention relates to a pharmaceutical composition comprising a compound of formula (I) as defined herein, or a pharmaceutically acceptable salt thereof, and optionally one or more pharmaceutically acceptable excipient(s) and/or carrier(s).

In a further aspect, the compound or the pharmaceutical composition of the present invention is for use as a medicament.

In yet another aspect, the present invention relates to a compound of formula (I) or a pharmaceutically acceptable salt thereof, or a pharmaceutical composition as described herein, for use in the treatment, amelioration or prevention of a serotonin 5-HT_{2A} receptor associated disease. For example, the present invention relates to a compound of formula (I) or a pharmaceutically acceptable salt thereof, or a pharmaceutical composition as described herein, for use in the treatment, amelioration or prevention of an anxiety disorder, attention deficit hyperactivity disorder (ADHD), depression, cluster headache, diminished drive, burn-out, bore-out, migraine, Parkinson's disease, pulmonary hypertension, schizophrenia, an eating disorder, nausea, vomiting, alcohol/drug dependency, or post-traumatic stress disorder (PTSD).

In a further aspect, the invention relates to the use of a compound of formula (I) or a pharmaceutically acceptable salt thereof for the preparation of a medicament for the treatment, amelioration or prevention of a serotonin 5-HT2A receptor associated disease. In particular, the invention relates to the use of a compound of formula (I) or a pharmaceutically acceptable salt thereof for the preparation of a medicament for the treatment, amelioration or prevention of an anxiety disorder, attention deficit hyperactivity disorder (ADHD), depression, cluster headache, diminished drive, burn-out, bore-out, migraine, Parkinson's disease, pulmonary hypertension, schizophrenia, an eating disorder, nausea, vomiting, alcohol/drug dependency, or post-traumatic stress disorder (PTSD).

In another aspect, the present invention provides a method of treating, ameliorating or preventing a serotonin 5-HT2A receptor associated disease, the method comprising administering a therapeutically effective amount of a compound of formula (I) or a pharmaceutically acceptable salt thereof, or a pharmaceutical composition as described herein, to a subject in need thereof. In particular, the invention provides a method of treating, ameliorating or preventing an anxiety disorder, attention deficit hyperactivity disorder (ADHD), depression, cluster headache, diminished drive, burn-out, bore-out, migraine, Parkinson's disease, pulmonary hypertension, schizophrenia, an eating disorder, nausea, vomiting, alcohol/drug dependency, or post-traumatic stress disorder (PTSD), the method comprising administering a therapeutically effective amount of a compound of formula (I) or a pharmaceutically acceptable salt thereof, or a pharmaceutical composition as described herein, to a subject in need thereof.

### BRIEF DESCRIPTION OF FIGURES

The following drawings are given by way of illustration only, and thus are not intended to limit the scope of the present invention.

FIG. 1: HPLC (top) and MS (bottom) spectrum for N,N-dimethyl-2-(4-(4,4,5,5-tetramethyl-1,3,2-dioxaborolan-2-yl)-1H indol-3-yl)ethan-1-amine (compound 1).

FIG. 2: HPLC (top) and MS (bottom) spectrum for 1-(3-(2-(dimethylamino)ethyl)-1H-indol-1-yl)propan-1-one (compound 2).

FIG. 3: HPLC (top) and MS (bottom) spectrum for 1-(3-(2-(dimethylamino)ethyl)-1H-indol-1-yl)-2,2,2-trifluoroethan-1-one (compound 3).

FIG. 4: HPLC (top) and MS (bottom) spectrum for 1-(3-(2-(dimethylamino)ethyl)-1H-indol-1-yl)-2,2-dimethylpropan-1-one (compound 4).

FIG. 5: HPLC (top) and MS (bottom) spectrum for cyclopropyl(3-(2-(dimethylamino)ethyl)-1H-indol-1-yl)methanone (compound 5).

FIG. 6: HPLC (top) and MS (bottom) spectrum for cyclobutyl(3-(2-(dimethylamino)ethyl)-1H-indol-1-yl)methanone (compound 6).

FIG. 7: HPLC (top) and MS (bottom) spectrum for 1-(3-(2-(dimethylamino)ethyl)-1H-indol-1-yl)-3-phenylpropan-1-one (compound 7).

FIG. 8: HPLC (top) and MS (bottom) spectrum for methyl 3-(2-(dimethylamino)ethyl)-1H-indole-1-carboxylate (compound 8).

FIG. 9: HPLC (top) and MS (bottom) spectrum for ethyl 3-(2-(dimethylamino)ethyl)-1H-indole-1-carboxylate (compound 9).

FIG. 10: HPLC (top) and MS (bottom) spectrum for isopropyl 3-(2-(dimethylamino)ethyl)-1H-indole-1-carboxylate (compound 10).

FIG. 11: HPLC (top) and MS (bottom) spectrum for tert-butyl 3-(2-(dimethylamino)ethyl)-1H-indole-1-carboxylate (compound 11).

FIG. 12: Degradation experiment for the boc carbamate compound tert-butyl 3-(2-(dimethylamino)ethyl)-1H-indole-1-carboxylate (compound 11) (top) and liberation of DMT (bottom).

FIG. 13: Degradation experiment for the pinacol boronic ester compound N,N-dimethyl-2-(4-(4,4,5,5-tetramethyl-1,3,2-dioxaborolan-2-yl)-1H-indol-3-yl)ethan-1-amine (compound 1) (top) and liberation of DMT (bottom).

FIG. 14: Degradation experiment for the TFA-amide compound 1-(3-(2-(dimethylamino)ethyl)-1H-indol-1-yl)-2,2,2-trifluoroethan-1-one (compound 3) (top) and liberation of DMT (bottom).

FIG. 15: Degradation experiment for the methyl-carbamate compound methyl 3-(2-(dimethylamino)ethyl)-1H-indole-1-carboxylate. (compound 8) (top) and liberation of DMT (bottom).

FIG. 16: Comparison charts: degradation of the pinacol boronic ester (compound 1), boc carbamate (compound 11), TFA amide (compound 3) and methyl carbamate (compound 8) prodrugs in 1% HCl (top); degradation of boc carbamate (compound 11) and methyl carbamate (compound 8) prodrugs at pH 7.5 or pH 8.0 (bottom).

FIG. 17: HPLC (top) and MS (bottom) spectrum for 2-(1-(cyclopropylsulfonyl)-1H-indol-3-yl)-N,N-dimethylethan-1-amine.

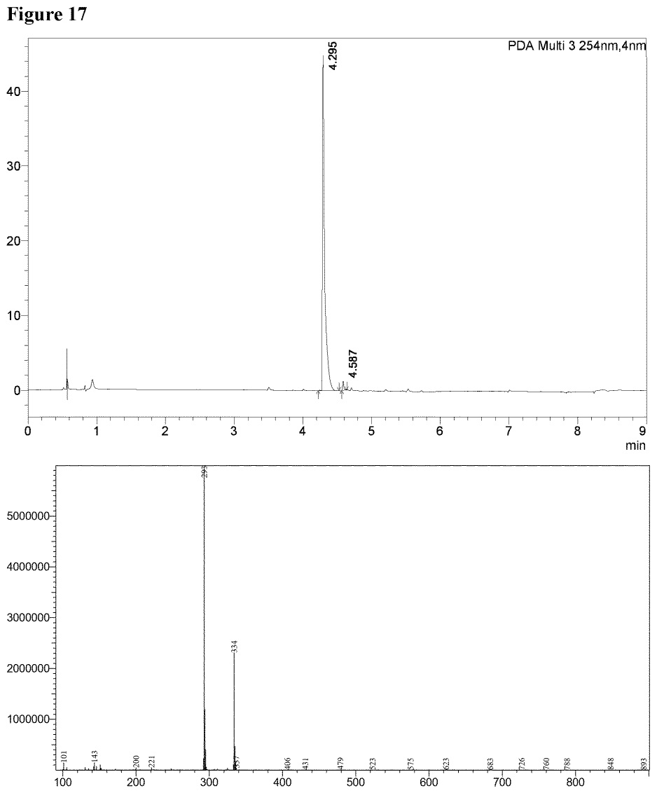

FIG. 18: HPLC (top) and MS (bottom) spectrum for 2-(1-(ethylsulfonyl)-1H-indol-3-yl)-N,N-dimethylethan-1-amine.

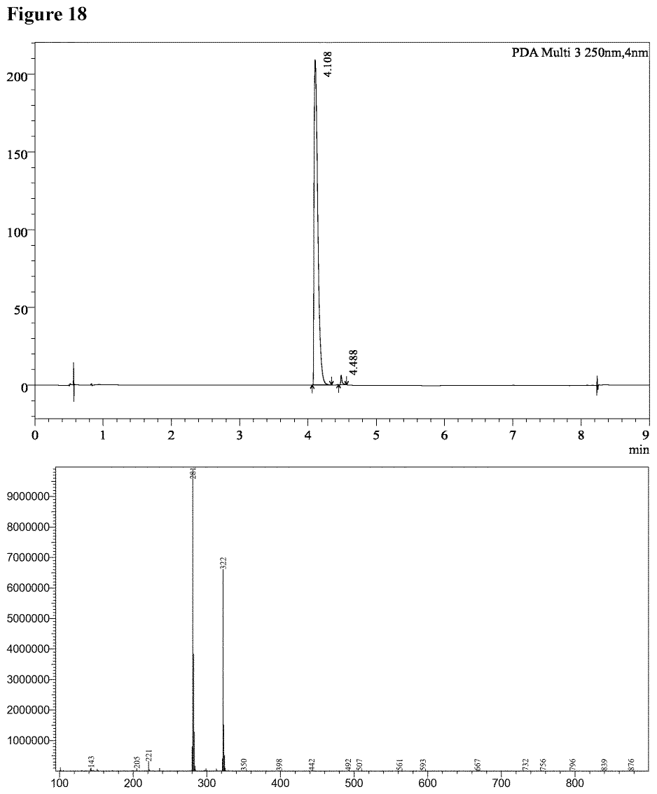

FIG. 19: HPLC (top) and MS (bottom) spectrum for 2-(1-(methylsulfonyl)-1H-indol-3-yl)-N,N-dimethylethan-1-amine.

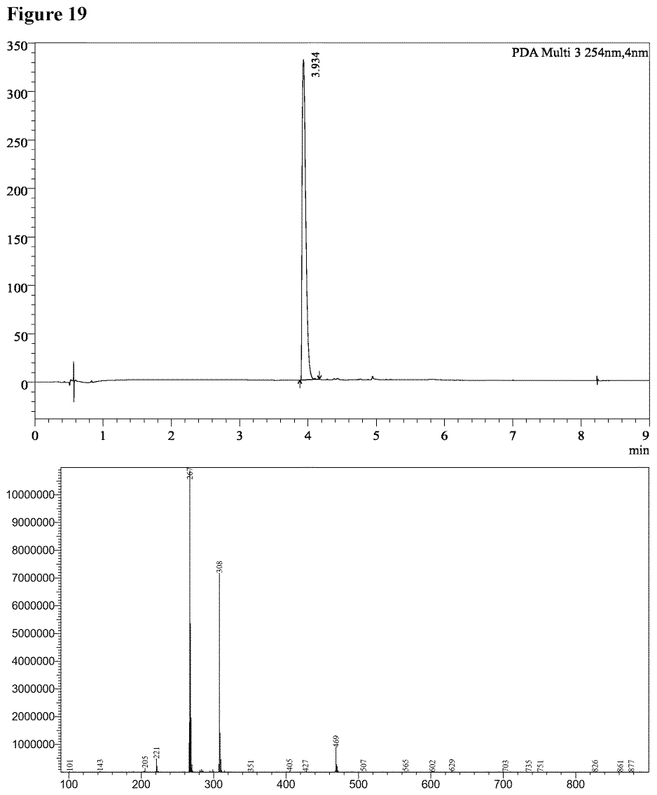

FIG. 20: Uptake inhibition assay at SERT performed for DMT, Example 1 and Example 4.

FIG. 21: Release assays performed for DMT, Example 1 and Example 4.

FIG. 22: Uptake inhibition assays performed for DMT, Example 13 and Example 15.

### DETAILED DESCRIPTION OF THE INVENTION

In a first aspect, the present invention relates to a compound according to the general formula (I), or a pharmaceutically acceptable salt thereof

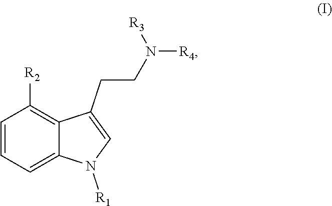

wherein:
R₁ is selected from the group consisting of hydrogen and

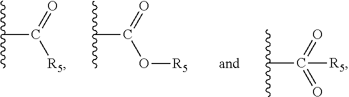

wherein R₅ is selected from C₁₋₁₂ alkyl (linear or branched), C₂₋₁₂ alkenyl (linear or branched), C₂₋₁₂ alkynyl (linear or branched), —CH₂—O—(CH₂CH₂O)ₘ—R₆, —O—(CH₂CH₂O)ₙ—R₆, —NH(CH₂CH₂O)ₚ—R₆, phenyl, benzyl, phenethyl, pyridyl, cycloalkyl, (cycloalkyl)alkyl, cycloalkenyl, (cycloalkenyl)alkyl, 5- or 6-membered heterocyclyl, (5- or 6-membered heterocyclyl)alkyl, and 9-fluorenylmethyl, wherein each one of the aforementioned groups is optionally substituted;
m, n and p are each independently an integer from 1 to 5;
R₆ is hydrogen or is selected from C₁₋₆ alkyl (linear or branched), aryl, heteroaryl, cycloalkyl, and heterocyclyl, wherein each one of the aforementioned groups is optionally substituted;
R₂ is selected from hydrogen and

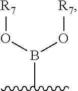

wherein each R₇ is independently selected from hydrogen and R₈, or wherein the two groups R₇, together with the —O—B—O— moiety that they are attached to, form a heterocyclyl which is optionally substituted;
wherein each R₈ is independently selected from C₁₋₁₂ alkyl (linear or branched), C₂₋₁₂ alkenyl (linear or branched), C₂₋₁₂ alkynyl (linear or branched), —CH₂—O—(CH₂CH₂O)ₘ—R₉, —O—(CH₂CH₂O)ₙ—R₉, —NH(CH₂CH₂O)ₚ—R₉, phenyl, benzyl, phenethyl, pyridyl, cycloalkyl, (cycloalkyl)alkyl, cycloalkenyl, (cycloalkenyl)alkyl, 5- or 6-membered heterocyclyl, (5- or 6-membered heterocyclyl)alkyl, and 9-fluorenylmethyl, wherein each one of the aforementioned groups is optionally substituted;
wherein m, n and p are as defined above;
wherein R₉ is hydrogen or is selected from C₁₋₆ alkyl, aryl, heteroaryl, cycloalkyl, and heterocyclyl, wherein each one of the aforementioned groups is optionally substituted;
R₃ is selected from hydrogen, methyl and ethyl;
R₄ is selected from hydrogen, methyl and ethyl; and
wherein at least one of R₁ and R₂ is not hydrogen.

In the following, exemplary embodiments of compounds according to the general formula (I) are described. It is to be understood that each exemplary embodiment is relevant on its own as well as in combination with other exemplary embodiments. Furthermore, it is to be understood that the examples in each case also apply to pharmaceutically acceptable salts of the compounds of the invention. It is further to be understood that for each of the exemplary embodiments described in the following, the variables R₁, R₂, R₃ and R₄ that are not explicitly defined in the description of said embodiment are as defined herein for the compound of formula (I) or for any specific embodiment disclosed herein.

Preferably, in the connection with the compounds of formula (I), R₁ is selected from

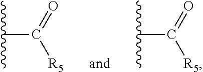

wherein R₅ is selected from C₁₋₁₂ alkyl (linear or branched), C₂₋₁₂ alkenyl (linear or branched), C₂₋₁₂ alkynyl (linear or branched), —CH₂—O—(CH₂CH₂O)ₘ—R₆, —O—(CH₂CH₂O)ₙ—R₆, —NH(CH₂CH₂O)ₚ—R₆, phenyl, benzyl, phenethyl, pyridyl, cycloalkyl, (cycloalkyl)alkyl, cycloalkenyl, (cycloalkenyl)alkyl, 5- or 6-membered heterocyclyl, (5- or 6-membered heterocyclyl)alkyl, and 9-fluorenylmethyl, wherein each one of the aforementioned groups is optionally substituted;
wherein m, n and p are each independently an integer from 1 to 5 (i.e., m, n and p are each independently selected from 1, 2, 3, 4 and 5);
wherein R₆ is hydrogen or is selected from C₁₋₆ alkyl, aryl, heteroaryl, cycloalkyl, and heterocyclyl, wherein each one of the aforementioned groups is optionally substituted.

More preferably, R₅ is selected from C₁₋₁₂ alkyl (linear or branched), C₂₋₁₂ alkenyl (linear or branched), C₂₋₁₂ alkynyl (linear or branched), phenyl, benzyl, phenethyl, pyridyl, cycloalkyl, (cycloalkyl)alkyl, cycloalkenyl, (cycloalkenyl)alkyl, 5- or 6-membered heterocyclyl, (5- or 6-membered heterocyclyl)alkyl, and 9-fluorenylmethyl, wherein each one of the aforementioned groups is optionally substituted.

Even more preferably, R₅ is selected from C₁₋₁₂ alkyl (linear or branched), phenethyl, and cycloalkyl, wherein each one of the aforementioned groups is optionally substituted.

Still more preferably, R₅ is selected from C₁₋₁₀ alkyl (linear or branched), phenethyl, and C₃-10 cycloalkyl, wherein each one of the aforementioned groups is optionally substituted.

Again more preferably, R₅ is selected from C₁₋₈ alkyl (linear or branched), phenethyl, and C₃₋₈ cycloalkyl, wherein each one of the aforementioned groups is optionally substituted.

Still even more preferably, R₅ is selected from C₁₋₆ alkyl (linear or branched), phenethyl, and C₃₋₆ cycloalkyl, wherein each one of the aforementioned groups is optionally substituted.

Again more preferably, R₅ is selected from C₁₋₄ alkyl (linear or branched), phenethyl, and C₃₋₄ cycloalkyl, wherein each one of the aforementioned groups is optionally substituted. For example, R₅ may be selected from methyl, ethyl, isopropyl, tert-butyl, trifluoromethyl, phenethyl, cyclopropyl, and cyclobutyl.

In one embodiment, R₁ may be

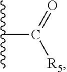

wherein R₅ is selected from C₁₋₁₂ alkyl (linear or branched), C₂₋₁₂ alkenyl (linear or branched), C₂₋₁₂ alkynyl (linear or branched), —CH₂—O—(CH₂CH₂O)ₘ—R₆, —O—(CH₂CH₂O)ₙ—R₆, —NH(CH₂CH₂O)ₚ—R₆, phenyl, benzyl, phenethyl, pyridyl, cycloalkyl, (cycloalkyl)alkyl, cycloalkenyl, (cycloalkenyl)alkyl, 5- or 6-membered heterocyclyl, (5- or 6-membered heterocyclyl)alkyl, and 9-fluorenylmethyl, wherein each one of the aforementioned groups is optionally substituted; preferably wherein R₅ is selected from C₁₋₁₂ alkyl (linear or branched), C₂₋₁₂ alkenyl (linear or branched), C₂₋₁₂ alkynyl (linear or branched), phenyl, benzyl, phenethyl, pyridyl, cycloalkyl, (cycloalkyl)alkyl, cycloalkenyl, (cycloalkenyl)alkyl, 5- or 6-membered heterocyclyl, (5- or 6-membered heterocyclyl)alkyl, and 9-fluorenylmethyl, wherein each one of the aforementioned groups is optionally substituted, more preferably wherein R₅ is selected from C₁₋₁₂ alkyl (linear or branched), phenethyl, and cycloalkyl, wherein each one of the aforementioned groups is optionally substituted; wherein m, n and p are each independently an integer from 1 to 5 (i.e., m, n and p are each independently selected from 1, 2, 3, 4 and 5); wherein R₆ is hydrogen or is selected from C₁₋₆ alkyl, aryl, heteroaryl, cycloalkyl, and heterocyclyl, wherein each one of the aforementioned groups is optionally substituted.

In one embodiment, R₁ may be

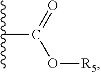

wherein R₅ is selected from phenyl, benzyl, phenethyl, pyridyl, cycloalkyl, (cycloalkyl)alkyl, cycloalkenyl, (cycloalkenyl)alkyl, 5- or 6-membered heterocyclyl, (5- or 6-membered heterocyclyl)alkyl, and 9-fluorenylmethyl, wherein each one of the aforementioned groups is optionally substituted.

Particularly preferred examples of R₁ include any of the following groups:

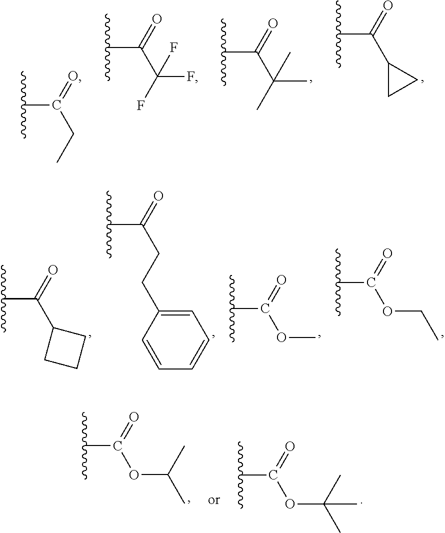

Even more preferred examples of R₁ include any of the following groups:

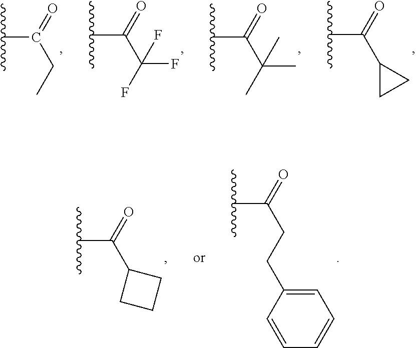

Particularly preferred groups R₁ are also individually disclosed in table A, as embodiments A-1 to A11.

TABLE A

| No. | R₁ |
|---|---|
| A-1 | H |
| A-2 | 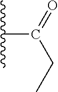 [SMILES: *C(=O)CC] |
| A-3 | 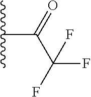 [SMILES: *C(=O)C(F)(F)F] |
| A-4 | 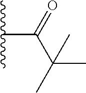 [SMILES: *C(=O)C(C)(C)C] |
| A-5 | 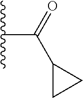 [SMILES: *C(=O)C1CC1] |
| A-6 | 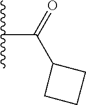 [SMILES: *C(=O)C1CCC1] |
| A-7 | 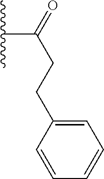 [SMILES: *C(=O)CCc1ccccc1] |
| A-8 | 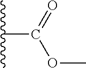 [SMILES: *C(=O)OC] |
| A-9 | 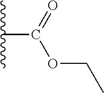 [SMILES: *C(=O)OCC] |
| A-10 | 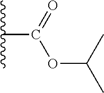 [SMILES: *C(=O)OC(C)C] |
| A-11 | 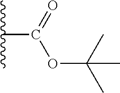 [SMILES: *C(=O)OC(C)(C)C] |

In one embodiment, R₁ is

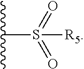

In this embodiment, it is particularly preferred that R₅ is C₁₋₁₂ alkyl which is optionally substituted, or R₅ is cycloalkyl which is optionally substituted.

Further, in connection with the compounds of formula (I), especially in connection with the compounds of formula (I) having a group R₁ as described hereinabove, including any of the particularly preferred groups R₁ as described in embodiments A-1 to A-11, R₂ is selected from hydrogen and

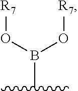

wherein each R₇ is independently selected from hydrogen and R₈, or wherein the two groups R₇, together with the —O—B—O— moiety that they are attached to, form a heterocyclyl which is optionally substituted;
wherein each R₈ is independently selected from C₁₋₁₂ alkyl (linear or branched), C₂₋₁₂ alkenyl (linear or branched), C₂₋₁₂ alkynyl (linear or branched), —CH₂—O—(CH₂CH₂O)ₘ—R₉, —O—(CH₂CH₂O)ₙ—R₉, —NH(CH₂CH₂O)ₚ—R₉, phenyl, benzyl, phenethyl, pyridyl, cycloalkyl, (cycloalkyl)alkyl, cycloalkenyl, (cycloalkenyl)alkyl, 5- or 6-membered heterocyclyl, (5- or 6-membered heterocyclyl)alkyl, and 9-fluorenylmethyl, wherein each one of the aforementioned groups is optionally substituted;
wherein m, n and p are each independently an integer from 1 to 5;
wherein R₉ is hydrogen or is selected from C₁₋₆ alkyl, aryl, heteroaryl, cycloalkyl, and heterocyclyl, wherein each one of the aforementioned groups is optionally substituted.

Preferably, each R₇ is independently selected from hydrogen and R₈, wherein each R₈ is independently selected from C₁₋₁₂ alkyl (linear or branched), C₂₋₁₂ alkenyl, C₂₋₁₂ alkynyl, phenyl, benzyl, phenethyl, pyridyl, cycloalkyl, (cycloalkyl)alkyl, cycloalkenyl and (cycloalkenyl)alkyl, wherein each one of the aforementioned groups is optionally substituted.

More preferably, each R₇ is independently selected from hydrogen and R₈, wherein each R₈ is independently a C₁₋₁₀ alkyl (linear or branched) which is optionally substituted.

Even more preferably, each R₇ is independently selected from hydrogen and R₈, wherein each R₈ is independently a C₁₋₈ alkyl (linear or branched) which is optionally substituted.

Still more preferably, each R₇ is independently selected from hydrogen and R₈, wherein each R₈ is independently a C₁₋₆ alkyl (linear or branched) which is optionally substituted.

Again more preferably, each R₇ is independently selected from hydrogen and R₈, wherein each R₈ is independently a C₁₋₄ alkyl (linear or branched) which is optionally substituted.

Alternatively, the two groups R₇, together with the —O—B—O— moiety that they are attached to (i.e., together with the O atoms that they are attached to and with the B atom to which said O atoms are attached), form a heterocyclyl which is optionally substituted.

In one embodiment, R₂ is selected from hydrogen and

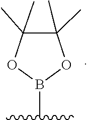

In one embodiment, R₂ is hydrogen. In one embodiment, R₂ is

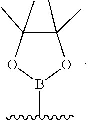

In connection with the compound of formula (I), especially in connection with the compounds of formula (I) including embodiments A-1 to A-11, the following embodiments according to table B are disclosed, with the proviso that at least one of R₁ and R₂ is not hydrogen.

TABLE B

| No. | R₂ |
|---|---|
| B-1 | H |
| B-2 | 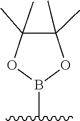 |

In the compound of formula (I), R₃ is selected from hydrogen, methyl and ethyl. Preferably, R₃ is selected from methyl and ethyl. More preferably, R₃ is methyl.

In the compound of formula (I), R₄ is selected from hydrogen, methyl and ethyl. Preferably, R₄ is selected from methyl and ethyl. More preferably, R₄ is methyl.

Furthermore, it is particularly preferred that both R₃ and R₄ are each methyl.

In addition, in connection with the compound of formula (I), the following combinations of meanings of R₁, R₂, R₃ and R₄ according to table C are disclosed.

TABLE C

| No. | R₁ | R₂ | R₃ | R₄ |
|---|---|---|---|---|
| C-1 | A-1 | B-2 | hydrogen | hydrogen |
| C-2 | A-2 | B-2 | hydrogen | hydrogen |
| C-3 | A-3 | B-2 | hydrogen | hydrogen |
| C-4 | A-4 | B-2 | hydrogen | hydrogen |
| C-5 | A-5 | B-2 | hydrogen | hydrogen |
| C-6 | A-6 | B-2 | hydrogen | hydrogen |
| C-7 | A-7 | B-2 | hydrogen | hydrogen |
| C-8 | A-8 | B-2 | hydrogen | hydrogen |
| C-9 | A-9 | B-2 | hydrogen | hydrogen |
| C-10 | A-10 | B-2 | hydrogen | hydrogen |
| C-11 | A-11 | B-2 | hydrogen | hydrogen |
| C-12 | A-2 | B-1 | hydrogen | hydrogen |
| C-13 | A-3 | B-1 | hydrogen | hydrogen |
| C-14 | A-4 | B-1 | hydrogen | hydrogen |
| C-15 | A-5 | B-1 | hydrogen | hydrogen |
| C-16 | A-6 | B-1 | hydrogen | hydrogen |
| C-17 | A-7 | B-1 | hydrogen | hydrogen |
| C-18 | A-8 | B-1 | hydrogen | hydrogen |
| C-19 | A-9 | B-1 | hydrogen | hydrogen |
| C-20 | A-10 | B-1 | hydrogen | hydrogen |
| C-21 | A-11 | B-1 | hydrogen | hydrogen |
| C-22 | A-1 | B-2 | methyl | hydrogen |
| C-23 | A-2 | B-2 | methyl | hydrogen |
| C-24 | A-3 | B-2 | methyl | hydrogen |
| C-25 | A-4 | B-2 | methyl | hydrogen |
| C-26 | A-5 | B-2 | methyl | hydrogen |
| C-27 | A-6 | B-2 | methyl | hydrogen |
| C-28 | A-7 | B-2 | methyl | hydrogen |
| C-29 | A-8 | B-2 | methyl | hydrogen |
| C-30 | A-9 | B-2 | methyl | hydrogen |
| C-31 | A-10 | B-2 | methyl | hydrogen |
| C-32 | A-11 | B-2 | methyl | hydrogen |
| C-33 | A-2 | B-1 | methyl | hydrogen |
| C-34 | A-3 | B-1 | methyl | hydrogen |
| C-35 | A-4 | B-1 | methyl | hydrogen |
| C-36 | A-5 | B-1 | methyl | hydrogen |
| C-37 | A-6 | B-1 | methyl | hydrogen |
| C-38 | A-7 | B-1 | methyl | hydrogen |
| C-39 | A-8 | B-1 | methyl | hydrogen |
| C-40 | A-9 | B-1 | methyl | hydrogen |
| C-41 | A-10 | B-1 | methyl | hydrogen |
| C-42 | A-11 | B-1 | methyl | hydrogen |
| C-43 | A-1 | B-2 | hydrogen | methyl |
| C-44 | A-2 | B-2 | hydrogen | methyl |
| C-45 | A-3 | B-2 | hydrogen | methyl |
| C-46 | A-4 | B-2 | hydrogen | methyl |
| C-47 | A-5 | B-2 | hydrogen | methyl |
| C-48 | A-6 | B-2 | hydrogen | methyl |
| C-49 | A-7 | B-2 | hydrogen | methyl |
| C-50 | A-8 | B-2 | hydrogen | methyl |
| C-51 | A-9 | B-2 | hydrogen | methyl |
| C-52 | A-10 | B-2 | hydrogen | methyl |
| C-53 | A-11 | B-2 | hydrogen | methyl |
| C-54 | A-2 | B-1 | hydrogen | methyl |
| C-55 | A-3 | B-1 | hydrogen | methyl |
| C-56 | A-4 | B-1 | hydrogen | methyl |
| C-57 | A-5 | B-1 | hydrogen | methyl |
| C-58 | A-6 | B-1 | hydrogen | methyl |
| C-59 | A-7 | B-1 | hydrogen | methyl |
| C-60 | A-8 | B-1 | hydrogen | methyl |
| C-61 | A-9 | B-1 | hydrogen | methyl |
| C-62 | A-10 | B-1 | hydrogen | methyl |
| C-63 | A-11 | B-1 | hydrogen | methyl |
| C-64 | A-1 | B-2 | methyl | methyl |
| C-65 | A-2 | B-2 | methyl | methyl |
| C-66 | A-3 | B-2 | methyl | methyl |
| C-67 | A-4 | B-2 | methyl | methyl |
| C-68 | A-5 | B-2 | methyl | methyl |
| C-69 | A-6 | B-2 | methyl | methyl |
| C-70 | A-7 | B-2 | methyl | methyl |
| C-71 | A-8 | B-2 | methyl | methyl |
| C-72 | A-9 | B-2 | methyl | methyl |
| C-73 | A-10 | B-2 | methyl | methyl |
| C-74 | A-11 | B-2 | methyl | methyl |
| C-75 | A-2 | B-1 | methyl | methyl |
| C-76 | A-3 | B-1 | methyl | methyl |
| C-77 | A-4 | B-1 | methyl | methyl |
| C-78 | A-5 | B-1 | methyl | methyl |
| C-79 | A-6 | B-1 | methyl | methyl |
| C-80 | A-7 | B-1 | methyl | methyl |
| C-81 | A-8 | B-1 | methyl | methyl |
| C-82 | A-9 | B-1 | methyl | methyl |
| C-83 | A-10 | B-1 | methyl | methyl |
| C-84 | A-11 | B-1 | methyl | methyl |
| C-85 | A-1 | B-2 | ethyl | hydrogen |
| C-86 | A-2 | B-2 | ethyl | hydrogen |
| C-87 | A-3 | B-2 | ethyl | hydrogen |
| C-88 | A-4 | B-2 | ethyl | hydrogen |
| C-89 | A-5 | B-2 | ethyl | hydrogen |
| C-90 | A-6 | B-2 | ethyl | hydrogen |
| C-91 | A-7 | B-2 | ethyl | hydrogen |
| C-92 | A-8 | B-2 | ethyl | hydrogen |
| C-93 | A-9 | B-2 | ethyl | hydrogen |
| C-94 | A-10 | B-2 | ethyl | hydrogen |
| C-95 | A-11 | B-2 | ethyl | hydrogen |
| C-96 | A-2 | B-1 | ethyl | hydrogen |
| C-97 | A-3 | B-1 | ethyl | hydrogen |
| C-98 | A-4 | B-1 | ethyl | hydrogen |
| C-99 | A-5 | B-1 | ethyl | hydrogen |
| C-100 | A-6 | B-1 | ethyl | hydrogen |
| C-101 | A-7 | B-1 | ethyl | hydrogen |
| C-102 | A-8 | B-1 | ethyl | hydrogen |
| C-103 | A-9 | B-1 | ethyl | hydrogen |
| C-104 | A-10 | B-1 | ethyl | hydrogen |
| C-105 | A-11 | B-1 | ethyl | hydrogen |
| C-106 | A-1 | B-2 | hydrogen | ethyl |
| C-107 | A-2 | B-2 | hydrogen | ethyl |
| C-108 | A-3 | B-2 | hydrogen | ethyl |
| C-109 | A-4 | B-2 | hydrogen | ethyl |
| C-110 | A-5 | B-2 | hydrogen | ethyl |
| C-111 | A-6 | B-2 | hydrogen | ethyl |
| C-112 | A-7 | B-2 | hydrogen | ethyl |
| C-113 | A-8 | B-2 | hydrogen | ethyl |
| C-114 | A-9 | B-2 | hydrogen | ethyl |
| C-115 | A-10 | B-2 | hydrogen | ethyl |
| C-116 | A-11 | B-2 | hydrogen | ethyl |
| C-117 | A-2 | B-1 | hydrogen | ethyl |
| C-118 | A-3 | B-1 | hydrogen | ethyl |
| C-119 | A-4 | B-1 | hydrogen | ethyl |
| C-120 | A-5 | B-1 | hydrogen | ethyl |
| C-121 | A-6 | B-1 | hydrogen | ethyl |
| C-122 | A-7 | B-1 | hydrogen | ethyl |
| C-123 | A-8 | B-1 | hydrogen | ethyl |
| C-124 | A-9 | B-1 | hydrogen | ethyl |
| C-125 | A-10 | B-1 | hydrogen | ethyl |
| C-126 | A-11 | B-1 | hydrogen | ethyl |
| C-127 | A-1 | B-2 | ethyl | methyl |
| C-128 | A-2 | B-2 | ethyl | methyl |
| C-129 | A-3 | B-2 | ethyl | methyl |
| C-130 | A-4 | B-2 | ethyl | methyl |
| C-131 | A-5 | B-2 | ethyl | methyl |
| C-132 | A-6 | B-2 | ethyl | methyl |
| C-133 | A-7 | B-2 | ethyl | methyl |
| C-134 | A-8 | B-2 | ethyl | methyl |
| C-135 | A-9 | B-2 | ethyl | methyl |
| C-136 | A-10 | B-2 | ethyl | methyl |
| C-137 | A-11 | B-2 | ethyl | methyl |
| C-138 | A-2 | B-1 | ethyl | methyl |
| C-139 | A-3 | B-1 | ethyl | methyl |
| C-140 | A-4 | B-1 | ethyl | methyl |
| C-141 | A-5 | B-1 | ethyl | methyl |
| C-142 | A-6 | B-1 | ethyl | methyl |
| C-143 | A-7 | B-1 | ethyl | methyl |
| C-144 | A-8 | B-1 | ethyl | methyl |
| C-145 | A-9 | B-1 | ethyl | methyl |
| C-146 | A-10 | B-1 | ethyl | methyl |
| C-147 | A-11 | B-1 | ethyl | methyl |
| C-148 | A-1 | B-2 | methyl | ethyl |
| C-149 | A-2 | B-2 | methyl | ethyl |
| C-150 | A-3 | B-2 | methyl | ethyl |
| C-151 | A-4 | B-2 | methyl | ethyl |
| C-152 | A-5 | B-2 | methyl | ethyl |
| C-153 | A-6 | B-2 | methyl | ethyl |
| C-154 | A-7 | B-2 | methyl | ethyl |
| C-155 | A-8 | B-2 | methyl | ethyl |
| C-156 | A-9 | B-2 | methyl | ethyl |
| C-157 | A-10 | B-2 | methyl | ethyl |
| C-158 | A-11 | B-2 | methyl | ethyl |
| C-159 | A-2 | B-1 | methyl | ethyl |
| C-160 | A-3 | B-1 | methyl | ethyl |
| C-161 | A-4 | B-1 | methyl | ethyl |
| C-162 | A-5 | B-1 | methyl | ethyl |
| C-163 | A-6 | B-1 | methyl | ethyl |
| C-164 | A-7 | B-1 | methyl | ethyl |
| C-165 | A-8 | B-1 | methyl | ethyl |
| C-166 | A-9 | B-1 | methyl | ethyl |
| C-167 | A-10 | B-1 | methyl | ethyl |
| C-168 | A-11 | B-1 | methyl | ethyl |
| C-169 | A-1 | B-2 | ethyl | ethyl |
| C-170 | A-2 | B-2 | ethyl | ethyl |
| C-171 | A-3 | B-2 | ethyl | ethyl |
| C-172 | A-4 | B-2 | ethyl | ethyl |
| C-173 | A-5 | B-2 | ethyl | ethyl |
| C-174 | A-6 | B-2 | ethyl | ethyl |
| C-175 | A-7 | B-2 | ethyl | ethyl |
| C-176 | A-8 | B-2 | ethyl | ethyl |
| C-177 | A-9 | B-2 | ethyl | ethyl |
| C-178 | A-10 | B-2 | ethyl | ethyl |
| C-179 | A-11 | B-2 | ethyl | ethyl |
| C-180 | A-2 | B-1 | ethyl | ethyl |
| C-181 | A-3 | B-1 | ethyl | ethyl |
| C-182 | A-4 | B-1 | ethyl | ethyl |
| C-183 | A-5 | B-1 | ethyl | ethyl |
| C-184 | A-6 | B-1 | ethyl | ethyl |
| C-185 | A-7 | B-1 | ethyl | ethyl |
| C-186 | A-8 | B-1 | ethyl | ethyl |
| C-187 | A-9 | B-1 | ethyl | ethyl |
| C-188 | A-10 | B-1 | ethyl | ethyl |
| C-189 | A-11 | B-1 | ethyl | ethyl |

A group of exemplary embodiments encompasses embodiment combinations C-64 to C-84, optionally embodiments C-64 and C-75 to C-84.

Specifically, the compound of formula (I) may be a compound selected from the group consisting of:

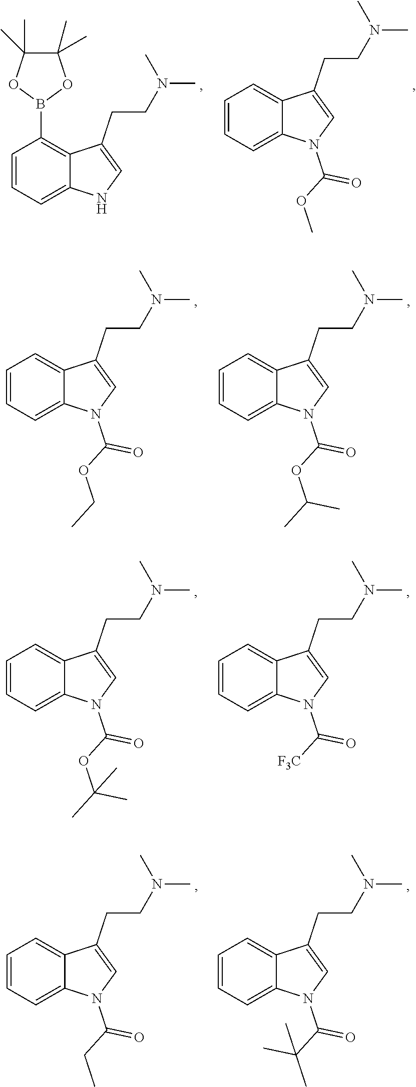

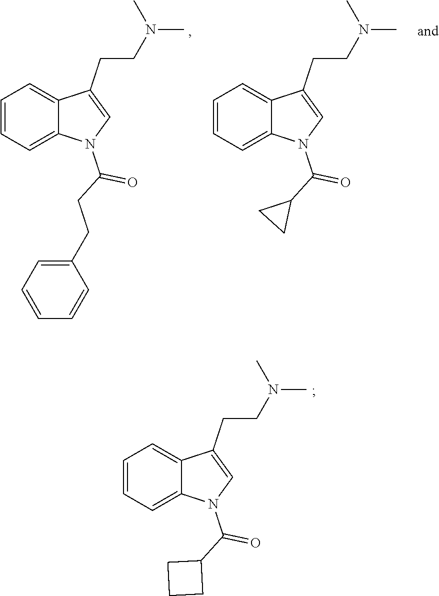 [SMILES: CN(C)CCc1cn(C(=O)CCc2ccccc2)c2ccccc12 ; CN(C)CCc1cn(C(=O)C2CC2)c2ccccc12 ; CN(C)CCc1cn(C(=O)C2CCC2)c2ccccc12]

or a pharmaceutically acceptable salt of any one of the above-depicted compounds.

Accordingly, the compound of formula (I) is preferably a compound selected from the group consisting of:
N,N-dimethyl-2-(4-(4,4,5,5-tetramethyl-1,3,2-dioxaborolan-2-yl)-1H-indol-3-yl)ethan-1-amine,
1-(3-(2-(dimethylamino)ethyl)-1H-indol-1-yl)propan-1-one,
1-(3-(2-(dimethylamino)ethyl)-1H-indol-1-yl)-2,2,2-trifluoroethan-1-one,
1-(3-(2-(dimethylamino)ethyl)-1H-indol-1-yl)-2,2-dimethylpropan-1-one,
cyclopropyl(3-(2-(dimethylamino)ethyl)-1H-indol-1-yl)methanone,
cyclobutyl(3-(2-(dimethylamino)ethyl)-1H-indol-1-yl)methanone,
1-(3-(2-(dimethylamino)ethyl)-1H-indol-1-yl)-3-phenylpropan-1-one,
methyl 3-(2-(dimethylamino)ethyl)-1H-indole-1-carboxylate,
ethyl 3-(2-(dimethylamino)ethyl)-1H-indole-1-carboxylate,
isopropyl 3-(2-(dimethylamino)ethyl)-1H-indole-1-carboxylate,
tert-butyl 3-(2-(dimethylamino)ethyl)-1H-indole-1-carboxylate,
2-(1-(cyclopropylsulfonyl)-1H-indol-3-yl)-N,N-dimethylethan-1-amine,
2-(1-(ethylsulfonyl)-1H-indol-3-yl)-N,N-dimethylethan-1-amine, and
2-(1-(methylsulfonyl)-1H-indol-3-yl)-N,N-dimethylethan-1-amine;
   or a pharmaceutically acceptable salt thereof.

Preferably, the compound of formula (I) is selected from
N,N-dimethyl-2-(4-(4,4,5,5-tetramethyl-1,3,2-dioxaborolan-2-yl)-1H-indol-3-yl)ethan-1-amine,
1-(3-(2-(dimethylamino)ethyl)-1H-indol-1-yl)propan-1-one,
1-(3-(2-(dimethylamino)ethyl)-1H-indol-1-yl)-2,2,2-trifluoroethan-1-one,
1-(3-(2-(dimethylamino)ethyl)-1H-indol-1-yl)-2,2-dimethylpropan-1-one,
cyclopropyl(3-(2-(dimethylamino)ethyl)-1H-indol-1-yl)methanone,
cyclobutyl(3-(2-(dimethylamino)ethyl)-1H-indol-1-yl)methanone,
1-(3-(2-(dimethylamino)ethyl)-1H-indol-1-yl)-3-phenylpropan-1-one,
methyl 3-(2-(dimethylamino)ethyl)-1H-indole-1-carboxylate,
ethyl 3-(2-(dimethylamino)ethyl)-1H-indole-1-carboxylate,
isopropyl 3-(2-(dimethylamino)ethyl)-1H-indole-1-carboxylate, and
tert-butyl 3-(2-(dimethylamino)ethyl)-1H-indole-1-carboxylate;
   or a pharmaceutically acceptable salt thereof.

As used herein, optional substituents attached to aryl, phenyl and heteroaryl rings each take the place of a hydrogen atom that would otherwise be present in any position on the aryl, phenyl or heteroaryl rings.

As used herein, halo or halogen groups include fluorine, chlorine, bromine and iodine. In other words, said halo (or halogen) group is preferably —F, —Cl, —Br or —I, more preferably —F, —Cl or —Br, even more preferably —F or —Cl, still more preferably —F.

As used herein, alkyl groups include straight-chain and branched-chain C₁₋₁₂ alkyl groups. Thus, whenever a reference is made to an alkyl group, it includes both straight chain and branched chain alkyl groups. Typical C₁₋₁₂ alkyl groups include methyl (Me), ethyl, n-propyl, n-butyl, n-pentyl, n-hexyl, n-heptyl, n-octyl, n-nonyl, n-decyl, n-undecyl, n-dodecyl, isopropyl, sec-butyl, tert-butyl, iso-butyl, iso-pentyl, neopentyl, 1-methylbutyl, 2-methylbutyl, 3-methylbutyl, 1,1-dimethylpropyl, 1,2-dimethylpropyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-methylhexyl, 2-methylhexyl, 3-methylhexyl, 4-methylhexyl, 5-methylhexyl, 1,2-dimethylpentyl, 1,3-dimethylpentyl, 1,2-dimethylhexyl, 1,3-dimethylhexyl, 3,3-dimethylhexyl, 1,2-dimethylheptyl, 1,3-dimethylheptyl, and 3,3-dimethylheptyl, among others. In one embodiment, C₁₋₁₂ alkyl groups are straight chain C₁₋₁₂ alkyl groups. In another embodiment, alkyl groups are selected from straight-chain and branched-chain C₁₋₁₀ alkyl groups, i.e., straight chain C₁₋₁₀ alkyl groups and branched chain C₃₋₁₀ alkyl groups. In another embodiment, alkyl groups are selected from straight-chain and branched-chain C₁₋₆ alkyl groups, i.e., straight chain C₁₋₆ alkyl groups and branched chain C₃₋₆ alkyl groups. Typical C₁₋₆ alkyl groups include methyl, ethyl, propyl, isopropyl, butyl, sec-butyl, tert-butyl, iso-butyl, pentyl, 3-pentyl, hexyl, among others. In one embodiment, alkyl groups are selected from straight-chain and branched-chain C₁₋₄ alkyl groups, i.e., straight chain C₁₋₄ alkyl groups and branched chain C₃₋₄ alkyl groups. Typical C₁₋₄ alkyl groups include methyl, ethyl, propyl, isopropyl, butyl, sec-butyl, tert-butyl, and iso-butyl. Typical C₁₋₂ alkyl groups include methyl and ethyl. In this application, a C₁₋₆ alkyl group refers to straight-chain and branched-chain C₁₋₆ alkyl groups, and a C₁₋₄ alkyl group refers to straight-chain and branched-chain C₁₋₄ alkyl groups, as defined above in this paragraph. In another embodiment, alkyl groups are selected from straight-chain and branched-chain C₇₋₁₂ alkyl groups.

As used herein, alkenyl groups include straight-chain and branched-chain C₂₋₁₂ alkenyl groups. As used herein, the term “C₂₋₁₂ alkenyl” as used by itself or as part of another group refers to straight chain and branched non-cyclic hydrocarbons having from 2 to 12 carbon atoms and including at least one carbon-carbon double bond. Representative C₂₋₁₂ alkenyl groups include vinyl, allyl, 1-butenyl, 2-butenyl, isobutenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, and the like. In one embodiment, C₂₋₁₂ alkenyl groups are C₂₋₁₀ alkenyl groups. In another embodiment, C₂₋₁₂ alkenyl groups are C₂₋₆ alkenyl groups. Typical C₂₋₆ alkenyl groups include ethenyl (i.e., vinyl), allyl, 1-butenyl, 2-butenyl, isobutenyl, 1-pentenyl, 2-pentenyl, 3-methyl-1-butenyl, 2-methyl-2-butenyl, 2,3-dimethyl-2-butenyl, 1-hexenyl, 2-hexenyl, and 3-hexenyl. Typical C₂₄ alkenyl groups include ethenyl, propenyl, isopropenyl, butenyl, and sec-butenyl.

As used herein, alkynyl groups include straight-chain and branched-chain C₂₋₁₂ alkynyl groups. As used herein, the term “C₂₋₁₂ alkynyl” as used by itself or as part of another groups refers to straight chain and branched non-cyclic hydrocarbons having from 2 to 12 carbon atoms and including at least one carbon-carbon triple bond. Representative straight chain and branched C₂₋₁₂ alkynyl groups include acetylenyl, propynyl, butyn-1-yl, butyn-2-yl, pentyn-1-yl, pentyn-2-yl, 3-methylbutyn-1-yl, pentyn-4-yl, hexyn-1-yl, hexyn-2-yl, hexyn-3-yl, and the like. In one embodiment, the C₂₋₁₂ alkynyl group is C₂₋₁₀ alkynyl group. In another embodiment, the C₂₋₁₂ alkynyl group is C₂₋₆ alkynyl group. Typical C₂₋₆ alkynyl groups include acetylenyl (i.e., ethynyl), propynyl, butyn-1-yl, butyn-2-yl, pentyn-1-yl, pentyn-2-yl, 3-methylbutyn-1-yl, pentyn-4-yl, and hexyn-1-yl groups. In another embodiment, the C₂₋₁₀ alkynyl group is a C₂₄ alkynyl group. Typical C₂₄ alkynyl groups include ethynyl, propynyl, butyn-1-yl, and butyn-2-yl groups.

As used herein, haloalkyl groups include any of the above-mentioned C₁₋₁₂ alkyl groups, optionally any of the above-mentioned C₁₋₆ alkyl groups, and optionally any of the above-mentioned C₁₋₄ alkyl groups, substituted by one or more fluorine, chlorine, bromine or iodine atoms (e.g., fluoromethyl, difluoromethyl, difluorochloromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups).

As used herein, cycloalkyl groups include saturated cyclic hydrocarbon groups containing 1, 2, or 3 rings having 3, 4, 5, 6, 7, 8, 9, 10, 11, or 12 carbon atoms (i.e., C₃₋₁₂ cycloalkyl) or the number of carbons designated. In one embodiment, the cycloalkyl has one or two rings. Exemplary cycloalkyl groups include cyclopropyl, cyclobutyl, cyclopentyl, cyclohexyl, cycloheptyl, cyclooctyl, norbornyl, decalin, and adamantyl. In another embodiment, the cycloalkyl is a C₃₋₆ cycloalkyl. Typical C₃₋₆ cycloalkyl groups include cyclopropyl, cyclobutyl, cyclopentyl and cyclohexyl.

As used herein, cycloalkenyl groups include partially unsaturated (i.e., containing one or two double bonds) cyclic hydrocarbon groups containing 1, 2, or 3 rings having 4, 5, 6, 7, 8, 9, 10, 11, or 12 carbon atoms (i.e., C₄-C₁₂ cycloalkenyl) or the number of carbons designated. In one embodiment, the cycloalkenyl has one or two rings. In another embodiment, the cycloalkenyl is a C₃₋₈ cycloalkenyl. In another embodiment, the cycloalkenyl is C₃₋₇ cycloalkenyl. In another embodiment, the cycloalkenyl is C₃₋₆ cycloalkenyl. In one embodiment, the cycloalkenyl group contains one double bond. Exemplary cycloalkenyl groups containing one double bond include cyclobutenyl, cyclopentenyl, cyclohexenyl, cycloheptenyl, cyclooctenyl, cyclononenyl, and cyclodecenyl. In another embodiment, the cycloalkenyl group contains two double bonds. Optionally, the cycloalkenyl groups containing two double bonds have 5, 6, 7, 8, 9, 10, 11, or 12 carbon atoms (i.e., C₅-C₁₂ cycloalkadienyl). Exemplary cycloalkenyl groups having two double bonds include cyclopentadienyl, cyclohexadienyl, cycloheptadienyl, cyclooctadienyl, cyclononadienyl, and cyclodecadienyl.

As used herein, alkoxy groups include oxygen substituted by one of the C₁₋₁₂ alkyl groups mentioned above (e.g., methoxy, ethoxy, propoxy, iso-propoxy, butoxy, tert-butoxy, iso-butoxy, sec-butoxy, pentyloxy, hexyloxy, heptyloxy, octyloxy, nonyloxy, decyloxy, undecyloxy, and dodecyloxy), optionally by one of the C₁₋₆ alkyl groups, and optionally by one of the C₁₋₄ alkyl groups.

As used herein, halo(C₁₋₆)alkoxy groups include oxygen substituted by one of the halo(C₁₋₆)alkyl groups mentioned above (e.g., fluoromethoxy, difluoromethoxy, trifluoromethoxy, and 2,2,2-trifluoroethoxy).

As used herein, (cycloalkyl)alkyl groups include any of the above-mentioned C₁₋₁₂ alkyl groups, and optionally any of the above-mentioned C₁₋₆ alkyl groups, substituted with any of the above-mentioned cycloalkyl groups (e.g., (cyclopropyl)methyl, 2-(cyclopropyl)ethyl, (cyclopropyl)propyl, (cyclobutyl)methyl, (cyclopentyl)methyl, and (cyclohexyl)methyl).

As used herein, (cycloalkenyl)alkyl groups include any of the above-mentioned C₁₋₁₂ alkyl groups, and optionally any of the above-mentioned C₁₋₆ alkyl groups, substituted with any of the above-mentioned cycloalkenyl groups (e.g., (cyclobutenyl)methyl, 2-(cyclobutenyl)ethyl, (cyclobutenyl)propyl, (cyclopentenyl)methyl, (cyclohexenyl)methyl, and (cyclopentadienyl)methyl).

As used herein, aryl groups include C₆₋₁₄ aryl, especially C₆₋₁₀ aryl. Typical C₆₋₁₄ aryl groups include phenyl (Ph), naphthyl, phenanthryl, anthracyl, indenyl, azulenyl, biphenyl, biphenylenyl, and fluorenyl groups, optionally phenyl, naphthyl, and biphenyl groups.

As used herein, arylalkyl groups include any of the above-mentioned C₁₋₁₂ alkyl groups, preferably any of the above-mentioned C₁₋₆ alkyl groups, substituted by any of the above-mentioned aryl groups (e.g., benzyl and phenethyl).

As used herein, arylalkenyl groups include any of the above-mentioned C₂₋₆ alkenyl groups substituted by any of the above-mentioned aryl groups (e.g., phenylethenyl).

As used herein, arylalkynyl groups include any of the above-mentioned C₂₋₆ alkynyl groups substituted by any of the above-mentioned aryl groups (e.g., phenylethynyl).

As used herein, aryloxy groups include oxygen substituted by one of the aryl groups mentioned above (e.g., phenoxy).

As used herein, aralkyloxy or arylalkoxy groups include oxygen substituted by one of the above-mentioned arylalkyl groups (e.g., benzyloxy).

As used herein, (arylalkoxy)carbonyl groups include a carbonyl group substituted by any of the above-mentioned arylalkoxy groups (e.g., (benzyloxy)carbonyl).

The terms “heterocyclyl”, “heterocycle” and “heterocyclo” are used herein to mean a saturated or partially unsaturated 3-7 membered monocyclic, or 7-10 membered bicyclic ring system, consisting of carbon atoms and from one to four heteroatoms independently selected from the group consisting of O, N, and S, wherein the nitrogen and sulfur heteroatoms can be optionally oxidized, the nitrogen can be optionally quaternized, and including any bicyclic group in which any of the above-defined heterocyclic rings is fused to a benzene ring, and wherein the heterocyclic ring can be substituted on a carbon atom or on a nitrogen atom if the resulting compound is stable. In one embodiment, the 3- to 7-membered monocyclic heterocyclic ring is either a saturated, or unsaturated non-aromatic ring. A 3-membered heterocycle contains 1 heteroatom, a 4-membered heterocycle can contain up to 2 heteroatoms, a 5-membered heterocycle can contain up to 4 heteroatoms, a 6-membered heterocycle can contain up to 4 heteroatoms, and a 7-membered heterocycle can contain up to 5 heteroatoms. Each heteroatom is independently selected from nitrogen, which can be quaternized; oxygen; and sulfur, including sulfoxide and sulfone. The 3- to 7-membered heterocycle can be attached via a nitrogen or carbon atom. A 7- to 10-membered bicyclic heterocycle contains from 1 to 4 heteroatoms independently selected from nitrogen, which can be quaternized; oxygen; and sulfur, including sulfoxide and sulfone. The 7- to 10-membered bicyclic heterocycle can be attached via a nitrogen or carbon atom. A monovalent radical derived from a heterocycle ring by removal of an H atom, and attached to another chemical moiety through the so formed empty valence, is referred to as heterocyclyl. Examples of the heterocyclyl include, but are not limited to, pyrrolidinyl, piperidinyl, piperazinyl, morpholinyl, imidazolinyl, pyrazolidinyl, tetrahydrofuranyl, oxazolidinyl, 2-oxooxazolidinyl, tetrahydrothienyl, imidazolidinyl, hexahydropyrimidinyl, and benzodiazepines. In one embodiment, the heterocyclyl is a 5- or 6-membered heterocyclyl. Typical 5-membered heterocyclyl groups include pyrrolidinyl, imidazolinyl, tetrahydrofuranyl, oxazolidinyl, 2-oxaoxazolidinyl, tetrahydrothienyl, and imidazolidinyl. Typical 6-membered heterocyclyl groups include piperidinyl, piperazinyl, morpholinyl, pyrazolidinyl, and hexahydropyrimidinyl.

Exemplary (heterocyclyl)alkyl groups include any of the above-mentioned C₁₋₁₀ alkyl groups, preferably any of the above-mentioned C₁₋₆ alkyl groups, substituted by any of the above-mentioned heterocyclyl groups (e.g., (pyrrolidin-2-yl)methyl, (pyrrolidin-1-yl)methyl, (piperidin-1-yl)methyl, (morpholin-4-yl)methyl, (2-oxooxazolidin-4-yl)methyl, 2-(2-oxooxazolidin-4-yl)ethyl, (2-oxo-imidazolidin-1-yl)methyl, (2-oxo-imidazolidin-1-yl)ethyl, and (2-oxo-imidazolidin-1-yl)propyl).

The term “heteroaryl” as used herein refers to groups having 5 to 14 ring atoms, with 6, 10 or 14 π electrons shared in a cyclic array, and containing carbon atoms and 1, 2, or 3 oxygen, nitrogen or sulfur heteroatoms, or 4 nitrogen atoms. In one embodiment, the heteroaryl group is a 5- to 10-membered heteroaryl group. In one embodiment, the heteroaryl group is a 5- or 6-membered heteroaryl having 1, 2, or 3 heteroatoms independently selected from O, N, and S. Examples of heteroaryl groups include thienyl, furyl, pyranyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, pyrimidinyl, thiazolyl, isothiazolyl, and isoxazolyl. A 5-membered heteroaryl can contain up to 4 heteroatoms. A 6-membered heteroaryl can contain up to 3 heteroatoms.

As used herein, the term “amino” or “amino group” refers to —NH₂.

Exemplary aminoalkyl groups include any of the above-mentioned C₁₋₁₂ alkyl groups, and optionally any of the above-mentioned C₁₋₆ alkyl groups, and optionally any of the above-mentioned C₁₋₄ alkyl groups substituted with one or more amino group.

Exemplary alkylamino and dialkylamino groups include NHR₁₀ and NR₁₀R₁₁, respectively, wherein R₁₀ and R₁₁ are each independently selected from a C₁₋₁₀ alkyl group, optionally a C₁₋₆ alkyl group, and optionally a C₁₋₄ alkyl group.

As used herein, the term “aminocarbonyl” refers to —C(═O)NH₂.

Exemplary alkylcarbonyl groups include a carbonyl group, i.e., —C(═O)—, substituted by any of the above-mentioned C₁₋₁₀ alkyl groups.

Exemplary arylcarbonyl groups include a carbonyl group substituted by any of the above-mentioned aryl groups (e.g., benzoyl).

Exemplary alkylcarbonyloxy or acyloxy groups include oxygen substituted by one of the above-mentioned alkylcarbonyl groups.

Exemplary alkylcarbonylamino or acylamino groups include any of the above-mentioned alkylcarbonyl groups attached to an amino nitrogen, such as methylcarbonylamino.

As used herein, the term “carboxamido” refers to a radical of formula —C(═O)NR₁₂R₁₃, wherein R₁₂ and R₁₃ are each independently hydrogen, optionally substituted C₁₋₁₀ alkyl, or optionally substituted aryl. Exemplary carboxamido groups include —CONH₂, —CON(H)CH₃, —CON(CH₃)₂, and —CON(H)Ph.

As used herein, the term “sulfonamido” refers to a radical of formula —SO₂NR₁₄R₁₅, wherein R₁₄ and R₁₅ are each independently hydrogen, optionally substituted C₁₋₁₀ alkyl, or optionally substituted aryl. Exemplary sulfonamido groups include —SO₂NH₂, —SO₂N(H)CH₃, and —SO₂N(H)Ph.

As used herein, the term “thiol” refers to —SH.

Exemplary mercaptoalkyl groups include any of the above-mentioned C₁₋₁₂ alkyl groups, and optionally any of the above-mentioned C₁₋₆ alkyl groups, substituted by a —SH group.

As used herein, the term “carboxy” refers to —COOH.

Exemplary carboxyalkyl groups include any of the above-mentioned C₁₋₁₂ alkyl groups, and optionally any of the above-mentioned C₁₋₆ alkyl groups, substituted by —COOH.

As used herein, the terms “hydroxyl” or “hydroxy” refer to —OH.

Exemplary hydroxyalkyl groups include any of the above-mentioned C₁₋₁₂ alkyl groups, optionally any of the above-mentioned C₁₋₆ alkyl groups, and optionally any of the above-mentioned C₁₋₄ alkyl groups, substituted by one or more hydroxy groups. Representative hydroxy(C₁₋₆)alkyl groups include hydroxymethyl, 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

As used herein, the term “cyano” refers to —CN.

As used herein, the term “nitro” refers to —NO₂.

As used herein, the term “ureido” refers to —NH—C(═O)—NH₂.

As used herein, the term “azido” refers to —N₃.

The term “about”, as used herein in connection with a measured quantity, refers to the normal variations in that measured quantity, as expected by the skilled artisan making the measurement and exercising a level of care commensurate with the objective of measurement and the precision of the measuring equipment. Typically, the term “about” includes the recited number ±10%. Thus, “about 10” means 9 to 11.

As used herein, the term “optionally substituted” refers to a group that may be unsubstituted or substituted. The optional substituents on “optionally substituted” groups, when not otherwise indicated, include one or more groups (typically 1, 2, or 3 groups) independently selected from halo, halo(C₁₋₆)alkyl, aryl, heterocyclyl, cycloalkyl, C₁₋₆ alkyl, C₂₋₆ alkenyl, C₂₋₆ alkynyl, aryl(C₁₋₆)alkyl, aryl(C₂₋₆)alkenyl, aryl(C₂₋₆)alkynyl, cycloalkyl(C₁₋₆)alkyl, heterocyclyl(C₁₋₆)alkyl, hydroxy(C₁₋₆)alkyl, amino(C₁₋₆)alkyl, carboxy(C₁₋₆)alkyl, alkoxy(C₁₋₆)alkyl, nitro, amino, ureido, cyano, alkylcarbonylamino, hydroxy, thiol, alkylcarbonyloxy, aryloxy, ar(C₁₋₆)alkyloxy, carboxamido, sulfonamido, azido, C₁₋₆ alkoxy, halo(C₁₋₆)alkoxy, carboxy, aminocarbonyl, (═O), and mercapto(C₁₋₆)alkyl. Optional substituents particularly include one or more groups (e.g., 1, 2 or 3 groups) independently selected from halo, halo(C₁₋₆)alkyl, hydroxy(C₁₋₆)alkyl, amino(C₁₋₆)alkyl, hydroxy, nitro, C₁₋₆ alkyl, C₁₋₆ alkoxy, halo(C₁₋₆)alkoxy, and amino. Unless explicitly indicated otherwise, where it is specified that a group is “optionally substituted”, it is preferred that 0, 1 or 2 substituents are present, more preferably 0 or 1 substituent, and even more preferably the corresponding group is unsubstituted.

Further disclosed are all pharmaceutically acceptable salt forms of the compounds of formula (I) which may be formed, e.g. by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g. sulfate or hydrogensulfate salts), nitrate salts, phosphate salts (such as, e.g. phosphate, hydrogenphosphate or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts.

Further pharmaceutically acceptable salts are described in the literature, e.g. in Stahl PH & Wermuth CG (eds.), “Handbook of Pharmaceutical Salts: Properties, Selection, and Use”, Wiley-VCH, 2002 and in the references cited therein.

Examples of a pharmaceutically acceptable salt of the compounds of the invention include, e.g. a fumarate salt, a maleate salt, an oxalate salt, a malate salt, or a tartrate salt. A further exemplary pharmaceutically acceptable salt is a fumarate salt. A further exemplary pharmaceutically acceptable salt is an oxalate salt.

The invention also specifically relates to the compounds of formula (I), including each one of the compounds described or exemplified herein, in non-salt form.

Compounds of the invention encompass any hydrated or solvated form, and any physical form, including any amorphous or crystalline forms. Solvates typically do not significantly alter the physiological activity or toxicity of the compounds, and as such may function as pharmacological equivalents. The term “solvate” as used herein is a combination, physical association and/or solvation of a compound of the present invention with a solvent molecule such as, e.g. a disolvate, monosolvate or hemisolvate, where the ratio of solvent molecule to compound of the present invention is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the disclosure may be present as solvated forms with a pharmaceutically acceptable solvent, such as water, methanol, ethanol, and the like, and it is intended that the invention includes both solvated and unsolvated forms of compounds of formula (I). One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al., J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1):Article 12 (2004), and A. L. Bingham et al., Chem. Commun.: 603-604 (2001). A typical, non-limiting, process of preparing a solvate would involve dissolving a compound of formula (I) in a desired solvent (organic, water, or a mixture thereof) at temperatures above about 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

Compounds of the disclosure can also be isotopically labeled (i.e., radiolabeled). Examples of isotopes that can be incorporated into the disclosed compounds include isotopes of hydrogen, carbon, nitrogen, oxygen, phosphorous, fluorine and chlorine, such as ²H, ³H, ¹¹C, ¹³C, ¹⁴C, ¹⁵N, ¹⁸O, ¹⁷O, ³¹P, ³²P, ³⁵S, ¹⁸F and ³⁶Cl, respectively. For example, the invention encompasses compounds of formula (I), in which one or more hydrogen atoms (or e.g. all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol % hydrogen 1 (¹H) and about 0.0156 mol % deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). The content of deuterium can be determined, e.g., using mass spectrometry or NMR spectroscopy.

### Methods of Making the Compounds of the Present Disclosure

In a further aspect, methods of making the compounds of the present invention are described. In one embodiment, a method for producing a DMT-boronic ester derivative as described herein is provided, comprising one or more of the following steps:
(a) preparing a solution of 4-bromo-N,N-dimethyltryptamine in a solvent I;
(b) adding an activating agent, optionally under a protective gas atmosphere;
(c) adding a derivatization agent and catalyst, optionally under a protective gas atmosphere;
(d) stirring the mixture, optionally under a protective gas atmosphere (e.g., for at least 3 hours), e.g., at 100° C.;
(e) filtering (e.g., paper) of the mixture;
(f) drying the filtrate, optionally at 40-60° C. and/or under vacuum or reduced pressure;
(g) obtaining a crude product;
(h) purifying the crude product, optionally by column chromatography;
(i) obtaining a DMT-boronic ester derivative according to the invention.

In one embodiment, in step (a), between 0.5 mmol and 3 mmol of 4-Bromo-N,N-dimethyltryptamine are suspended in 8 ml to 30 ml of solvent I, wherein solvent I is selected from tetrahydrofuran, dioxane, 2-methyltetrahydrofuran, dimethoxyethane and toluene. This can be done at a temperature between 25° C. and 150° C., optionally at a temperature between 50° C. and 120° C., for instance at 105° C. (378.15 Kelvin).

In one embodiment, in step (b), between 1.5 mmol and 9 mmol of an activating agent are added, such as, e.g., a nitrogen base or an inorganic base. In this case, in exemplary embodiments, the inorganic base is selected from potassium acetate, potassium carbonate, potassium phenolate, potassium tert-butoxide and potassium 2-ethylhexanoate. The nitrogen base is selected from triethylamine, diisopropyl ethylamine, pyridine, and 4-dimethyl aminopyridine. The suspension obtained is aerated with protective gas.

In one embodiment, in step (c), between 0.75 mmol and 4.5 mmol of a derivatization agent bis(pinacolato)diboron and a catalyst is added, wherein the catalyst is selected from Bis(triphenylphosphine)palladium(II) dichloride, tetrakis(triphenylphosphine)palladium(0), [1,1′-bis(diphenylphosphino)ferrocene]dichloropalladium(II) and [1,3-bis(diphenylphosphino)propane]dichloronickel(II).

In one embodiment, in step (d), the mixture is stirred between 12 and 36 hours at 105° C. under protective gas atmosphere. In one embodiment, it is stirred for at least 20 hours and up to 30 hours; and/or at 105° C. under protective gas atmosphere.

In one embodiment, in step (e), the crude reaction mixture, e.g. resulting from step (c) is filtered through a paper filter.

In one embodiment, in step (f), the filtrate is dried. One example is drying with a rotary evaporator at a temperature between 35° C. and 60° C. under vacuum or reduced pressure of 20-60 mbar. In one embodiment, the temperature is 45° C. and the vacuum is 40 mbar.

A crude product resulting from step (f) may contain the DMT-boronic ester derivative according to the invention.

In a further embodiment, in step (h), the crude product is purified. The purification can be conducted by a column purification, e.g., over silica using acetone as the eluent. Other column materials and eluents are known in the art can also be used. With this method, yields of more than 70 wt.-% (gravimetric determination of the amount of the end product, relative to the initial materials) can be achieved.

The purified product resulting from above steps (a) to (h) contains the pure DMT-boronic ester derivative according to the invention.

Other methods of providing a DMT-boronic ester derivative according to the invention are provided in the examples or will be apparent to the person skilled in the art.

A method for producing DMT carbamate/amide derivatives as described herein is also provided, comprising one or more of the following steps:
(a) preparing a solution of DMT in a solvent I;
(b) adding an activating agent, optionally under a protective gas atmosphere;
(c) adding a derivatization agent;
(d) stirring the mixture, optionally under a protective gas atmosphere (e.g., for at least 12 hours);
(e) stopping the reaction, optionally by dilution with solvent (e.g., solvent I from step (a));
(f) extracting, e.g., with water and saturated saline solution;
(g) drying the organic phase, e.g., over a desiccant at 40-60° C. under vacuum or reduced pressure;
(h) obtaining a crude product;
(i) purifying the crude product, e.g., by column chromatography and/or recrystallization;
(j) obtaining the DMT derivatives according to the invention.

In one embodiment, the method for producing DMT carbamate/amide derivates comprises step (a), wherein between 0.25 mmol and 3.3 mmol of N,N-dimethyltryptamine are dissolved in 4 ml to 35 ml of solvent I, wherein solvent I is selected from chloroform, tetrahydrofuran, dioxane, 2-methyltetrahydrofuran and dichloromethane. This can be done at a temperature between 25° C. and 100° C., optionally at a temperature between 40° C. and 60° C., for example at 50° C. (323.15 Kelvin).

In one embodiment, in step (b), between 0.3 mmol and 9 mmol of an activating agent are added, such as, e.g., a nitrogen base. In this case, in exemplary embodiments, the nitrogen base is selected from triethylamine, diisopropyl ethylamine, pyridine, diazabicycloundecene, diazabicyclononene and 4-dimethyl aminopyridine. It is also possible to use a deprotonating agent such as n-butyllithium (n-BuLi), lithium isopropylamide (LDA) and sodium hydride.

In one embodiment, in step (c), a derivatization agent (e.g., between 0.38 mmol and 13 mmol) are added (e.g., dropwise through a septum), wherein the derivatization agent is selected from methyl chloroformate, ethyl chloroformate, di-tert-butyl pyrocarbonate, isopropylchloroformate, trifluoroacetic anhydride, propionic anhydride, 3-phenylpropionyl chloride, trimethylacetic anhydride, cyclopropanecarboxylic acid chloride and cyclobutanecarboxylic acid chloride.

In one embodiment, in step (d), the mixture is stirred between 1 hour (e.g., by use of n-butyllithium) and 7 days at 0° C. (e.g., by use of n-butyllithium) or 40-60° C. under protective gas atmosphere.

In one embodiment, in step (e), the reaction is stopped by adding between 20 ml and 180 ml of the solvent I from step (a).

In one embodiment, in step (f), extraction is performed with between 50 ml and 150 ml of water. In one embodiment, subsequent extraction with between 50 ml and 150 ml saturated saline solution is performed.

In one embodiment, in step (g), the mixture is dried. For instance, drying with a desiccant at a temperature between 35° C. and 60° C. and a vacuum (reduced pressure) of 30-60 mbar is disclosed. Exemplary desiccants are anhydrous calcium chloride, anhydrous sodium carbonate, anhydrous potassium carbonate, anhydrous sodium sulfate, anhydrous magnesium sulphate, or anhydrous calcium sulfate. In one embodiment, the desiccant is anhydrous MgSO4, the temperature is 45° C. and the vacuum is 40 mbar.

The crude product resulting from step (g) may contain the DMT-carbamate/-amide derivative according to the invention.

In one embodiment, in step (i), the crude product is further purified. The purification can be conducted by a column purification over silica using acetone as the eluent. Other column materials and eluents are known in the art can also be used. With this method, yields of more than 30 wt-% (gravimetric determination of the amount of the end product, relative to the initial materials) can be achieved.

The product resulting from step (i) may contain the DMT-carbamate/-amide derivative according to the invention.

Other methods of providing a DMT-carbamate/-amide derivative according to the invention are provided in the examples or will be apparent to the person skilled in the art.

### Therapeutic Uses of Compounds of the Present Invention

Compounds of the present invention can act as prodrugs and thereby exhibit one or more advantages over the corresponding parent drugs, e.g., DMT or DMT analogs. The compounds of the invention exhibit beneficial pharmacological properties. For example, the compounds of the invention are pharmacologically released, taken up and metabolized in the human body with improved pharmacokinetics as compared to DMT and DMT analogs, respectively. The potential for addiction of psychotropic substances can be related to the rapid increase in their concentration upon uptake. Therefore, from a pharmaceutical point of view, active compounds that cause a slow increase in the initial concentration can be advantageous. The chemical modification of DMT or analogs thereof in the form of the compounds described herein may reduce the potential for abuse because a rapid “flooding” of the active compound is suppressed.

Further, due to their chemical modification and the resulting stability towards MAO degradation, the compounds according to the invention may exhibit a higher efficacy than DMT or DMT analogs, respectively, which makes these compounds particularly suitable for therapeutic use.

Moreover, without being bound by theory, the compounds provided herein can exert their effect in the organism after endogenous metabolization into an actually active compound such as DMT or DMT analogs, which may result in a longer-lasting effect (“depot effect”). Thus, in some embodiments, the compounds disclosed herein can be selectively cleaved by certain endogenous enzymes. In some embodiments, the compounds disclosed herein, as, for instance, N-phenylpropionylamide, can be selectively cleaved by chymotrypsin.

A more constant and uniform release of the actually active compound, such as DMT or DMT analogs, in the organism can also contribute to the reduction of side effects. Thus, in a further aspect, the present invention relates to a pharmaceutical composition comprising a compound of formula (I) as defined herein and optionally one or more pharmaceutically acceptable excipient(s) and/or carrier(s).

The compounds provided herein may be administered as compounds per se or may be formulated as pharmaceutical or pharmacological compositions or medicaments. The pharmaceutical compositions or medicaments may optionally comprise one or more pharmaceutically acceptable excipients, such as carriers, diluents, fillers, disintegrants, lubricating agents, binders, colorants, pigments, stabilizers, preservatives, and/or antioxidants.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are an exemplary dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

In a further aspect, the compound of the present invention or the pharmaceutical composition comprising the same is for use as a medicament.

In another aspect, the present invention relates to a compound of formula (I) as defined herein or a pharmaceutically acceptable salt thereof, or a pharmaceutical composition as defined herein, for use in the treatment, amelioration or prevention of a serotonin 5-HT_{2A} receptor associated disease.

In another aspect, the present invention relates to a compound of formula (I) as defined herein or a pharmaceutically acceptable salt thereof, or a pharmaceutical composition as defined herein, for use in the treatment, amelioration or prevention of an anxiety disorder, attention deficit hyperactivity disorder (ADHD), depression, cluster headache, diminished drive, burn-out, bore-out, migraine, Parkinson's disease, pulmonary hypertension, schizophrenia, an eating disorder, nausea, vomiting, alcohol/drug dependency, or post-traumatic stress disorder (PTSD), preferably an anxiety disorder, attention deficit hyperactivity disorder (ADHD), depression, cluster headache, diminished drive, burn-out, bore-out, migraine, Parkinson's disease, pulmonary hypertension, schizophrenia, an eating disorder, nausea, vomiting, or alcohol/drug dependency.

Selection of exemplary compounds provided by formula (I) for in vivo applications can be performed by methods known to those skilled in the art. By way of example, prodrug activity in animals, such as rats, can be investigated by determining the concentrations of the prodrug and active compound in the relevant target tissues following administration. For this purpose, intravenous injection or oral ingestion, may be chosen as exemplary method of administrating the prodrug of formula (I) to be investigated. The conversion rate of the selected prodrug into its breakdown compound, DMT or DMT analog, may be assessed by measuring the concentration of both compounds in brain and blood tissues using HPLC assay at different time points following administration.

In accordance with the foregoing, the present invention in particular relates to the following items:
1. Compound according to the general formula (I), optionally in the form of a pharmaceutically acceptable salt thereof

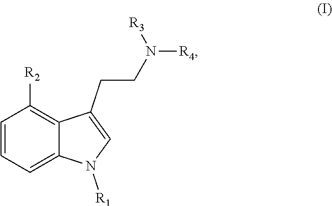

wherein:
   R₁ is selected from the group consisting of hydrogen,

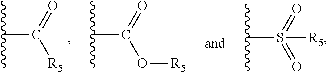

wherein R₅ is a substituted or unsubstituted group selected from C₁₋₁₂ alkyl (linear or branched), C₂₋₁₂ alkenyl (linear or branched), C₂₋₁₂ alkynyl (linear or branched), —CH₂—O—(CH₂CH₂O)ₘ—R₆, —O—(CH₂CH₂O)ₙ—R₆, —NH(CH₂CH₂O)ₚ—R₆, phenyl, benzyl, phenethyl, pyridyl, cycloalkyl, (cycloalkyl)alkyl, cycloalkenyl, (cycloalkenyl)alkyl, 5- or 6-membered heterocycle, (5- or 6-membered heterocycle)alkyl, and 9-fluorenylmethyl, R₆ is hydrogen or a substituted or unsubstituted group selected from C₁₋₆ alkyl (linear or branched), aryl, heteroaryl, cycloalkyl, and heterocycle;
   R₂ is selected from hydrogen and

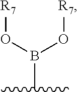

wherein R₇ is selected from hydrogen and R₈, or wherein the two R₇ form a heterocycle, and wherein R₈ is a substituted or unsubstituted group selected from C₁₋₁₂ alkyl (linear or branched), C₂₋₁₂ alkenyl (linear or branched), C₂₋₁₂ alkynyl (linear or branched), —CH₂—O—(CH₂CH₂O)ₘ—R₉, —O—(CH₂CH₂O)ₙ—R₉, —NH(CH₂CH₂O)ₚ—R₉, phenyl, benzyl, phenethyl, pyridyl, cycloalkyl, (cycloalkyl)alkyl, cycloalkenyl, (cycloalkenyl)alkyl, 5- or 6-membered heterocycle, (5- or 6-membered heterocycle)alkyl, and 9-fluorenylmethyl,
   wherein R₉ is hydrogen or a substituted or unsubstituted group selected from C₁₋₆ alkyl, aryl, heteroaryl, cycloalkyl, and heterocycle;
   R₃ is selected from hydrogen, methyl and ethyl;
   R₄ is selected from hydrogen, methyl and ethyl; and
   wherein at least one of R₁ and R₂ is not hydrogen.
2. The compound according to item 1, wherein R₂ is

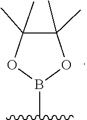

3. The compound according to item 1 or 2, wherein R₁ is

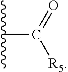

4. The compound according to item 1 or 2, wherein R₁ is

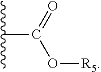

5. The compound according to item 1 or 2, wherein R₁ is selected from

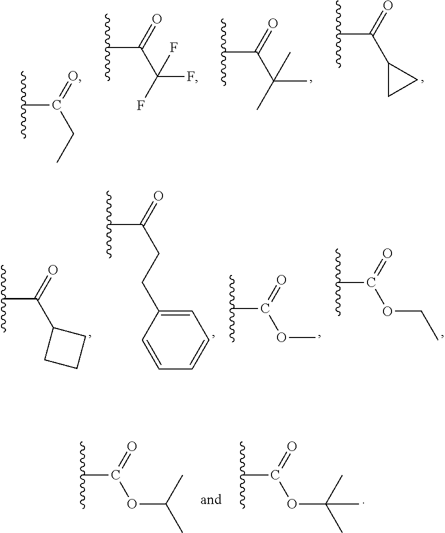

6. The compound according to item 1, wherein the compound is selected from the group consisting of:
N,N-dimethyl-2-(4-(4,4,5,5-tetramethyl-1,3,2-dioxaborolan-2-yl)-1H-indol-3-yl)ethan-1-amine,
1-(3-(2-(dimethylamino)ethyl)-1H-indol-1-yl)propan-1-one,
1-(3-(2-(dimethylamino)ethyl)-1H-indol-1-yl)-2,2,2-trifluoroethan-1-one,
1-(3-(2-(dimethylamino)ethyl)-1H-indol-1-yl)-2,2-dimethylpropan-1-one cyclopropyl(3-(2-(dimethylamino)ethyl)-1H-indol-1-yl)methanone,
cyclobutyl(3-(2-(dimethylamino)ethyl)-1H-indol-1-yl)methanone,
1-(3-(2-(dimethylamino)ethyl)-1H-indol-1-yl)-3-phenylpropan-1-one,
methyl 3-(2-(dimethylamino)ethyl)-1H-indole-1-carboxylate,
ethyl 3-(2-(dimethylamino)ethyl)-1H-indole-1-carboxylate,
isopropyl 3-(2-(dimethylamino)ethyl)-1H-indole-1-carboxylate, and
tert-butyl 3-(2-(dimethylamino)ethyl)-1H-indole-1-carboxylate;
   or a pharmaceutically acceptable salt thereof.
   7. A pharmaceutical composition comprising a compound of any one of items 1 to 6 and optionally one or more pharmaceutically acceptable excipient(s) and/or carrier(s).
   8. The compound of any one of items 1 to 6 or the pharmaceutical composition of item 7 for use as a medicament.
   9. The compound of any one of items 1 to 6 or the pharmaceutical composition of item 7 for use in the treatment, amelioration or prevention of a serotonin 5-HT_{2A} receptor associated disease.
   10. The compound of any one of items 1 to 6 or the pharmaceutical composition of item 7 for use in the treatment, amelioration or prevention of an anxiety disorder, attention deficit hyperactivity disorder (ADHD), depression, cluster headache, diminished drive, burn-out, bore-out, migraine, Parkinson's disease, pulmonary hypertension, schizophrenia, an eating disorder, nausea, vomiting or alcohol/drug dependency.

The present invention is further illustrated by the following examples.

### EXAMPLES

### General Experimental Methods for Producing the Compounds of the Present Invention

“HPLC” means high-performance liquid chromatography.

“TLC” means thin layer chromatography

The LC-MS measurements for all examples described herein were performed as in the following:

### Lc-System:

Running time: 9.0 min
Column: Supelco Titan C18 1.9 micrometer; dimension 100×2.1 mm
Oven temperature: 40° C.
UV-detection: 215-400 nm
Solvent A: Water
Solvent B: Acetonitrile

### Gradient Conditions:

| | Flow | A | B | Time |
|---|---|---|---|---|
| 1 | 0.7 ml/min | 98% | 2% | |
| 2 | 0.7 ml/min | 98% | 2% | 1.00 min |
| 3 | 0.7 ml/min | 10% | 90% | 8.00 min |
| 4 | 0.7 ml/min | 2% | 98% | 8.02 min |
| 5 | 0.7 ml/min | 3% | 98% | 8.30 min |
| 6 | 0.7 ml/min | 98% | 2% | 8.40 min |

### MS System:

Running time: 9.0 min
Acquisition mode: Scan positive
Start m/z: 90 End m/z: 900
Scan speed: 10000 u/see
Event time: 0.1 sec

### Example 1: Method of production of N,N-dimethyl-2-(4-(4,4,5,5-tetramethyl-1,3,2-dioxaborolan-2-yl)-1H-indol-3-yl)ethan-1-amine

4-bromo-N,N-dimethyltryptamine (3 mmol, 801 mg) was dissolved in dioxane (30 mL) at 25° C. Potassium acetate (9 mmol, 882 mg) was added. Then, bis(triphenylphosphine)palladium(II) dichloride (0.15 mmol, 105 mg) was added. Consequently, bis(pinacolato)diboron (4.5 mmol, 1.14 g) was added and the flask was aerated with argon. The reaction mixture was stirred at 105° C. for 4 hours under argon.

In LC/MS at a wavelength of 274 nm, a sample of the reaction mixture showed almost complete reaction of the starting material. According to HPLC, 32% of the product could be quantified.

The brown suspension was stirred at 105° C. for additional 18 hours under argon. The reaction mixture was filtered and the filtrate was concentrated on a rotary evaporator at 42° C. and subsequently dried at up to 10 mbar.

The crude product was taken up in 2 mL acetone and purified via column chromatography over silica using acetone as the eluent. Subsequently, the corresponding fractions were collected and slowly concentrated on the rotary evaporator to yield 300 mg of brown solid. Additionally, fractions with lower purity could be collected (430 mg).

### Example 2: Method of production of 1-(3-(2-(dimethylamino)ethyl)-1H-indol-1-yl)proyan-1-one

N,N-Dimethyltryptamine (1 mmol, 188 mg) was dissolved in tetrahydrofuran (12 mL) at 4° C. and was then aerated with argon. N-Butyl lithium solution in hexane (1.15 mmol, 0.46 ml at 2.5 M) added dropwise through septum. After the addition of propionic anhydride (1.5 mmol/0.19 mL) through the septum, the reaction mixture was stirred at 4° C. for an additional 40 minutes.

According to TLC and HPLC, nearly complete consumption of starting material has occurred.

The reaction mixture was evaporated and the resulting residue dissolved in dichloromethane (60 mL) and extracted with water (30 mL) and saturated saline solution (30 mL). The organic phase was dried over magnesium sulfate and evaporated to yield a yellow oil (350 mg) as a crude product.

The crude product was taken up in dichloromethane and purified via column chromatography over silica using a mixture of dichloromethane and methanol as the eluent. Subsequently, the corresponding fractions were collected and slowly concentrated on the rotary evaporator.

The purified product was dissolved in acetone. A colorless fumarate salt (110 mg) has obtained after addition of fumaric acid.

### Example 3: Method of production of 1-(3-(2-(dimethylamino)ethyl)-1H-indol-1-yl)-2,2,2-trifluoroethan-1-one

N,N-Dimethyltryptamine (1.5 mmol, 282 mg) was dissolved in chloroform (8 mL) at 25° C. N,N-dimethylaminopyridine (4.5 mmol, 549 mg) was added and the flask was aerated with argon. After dropwise addition of trifluoroacetic anhydride (3 mmol/0.42 mL) through the septum, the reaction mixture was stirred at 25° C. for 20 hours.

In LC/MS at a wavelength of 274 nm, a sample of the reaction mixture showed 80% conversion. Trifluoroacetic anhydride (1.4 mmol, 0.2 mL) was subsequently added and stirred at 25 C for 2 hours. The reaction mixture was diluted with dichloromethane and extracted with water (50 mL) and saturated saline solution (50 mL). The organic phase was dried over magnesium sulfate and evaporated to yield a reddish solid (550 mg).

After recrystallization of the crude product in acetone, colorless crystals (138 mg) were obtained.

### Example 4: Method of production of 1-(3-(2-(dimethylamino)ethyl)-1H-indol-1-yl)-2,2-dimethylpropan-1-one

N,N-Dimethyltryptamine (1.1 mmol, 200 mg) was dissolved in tetrahydrofuran (12 mL) at 4° C. and then was aerated with argon. An n-butyl lithium solution in hexane (1.17 mmol, 0.47 mL at 2.5 M) was added dropwise through a septum. After addition of pivaloyl anhydride (1.5 mmol/0.31 mL) through the septum, the reaction mixture was stirred at 4° C. for an additional 35 minutes.

According to TLC and HPLC, nearly complete consumption of the starting material has occurred.

The reaction mixture was evaporated, the resulting residue was dissolved in dichloromethane (70 mL) and extracted with water (30 mL) and saturated saline solution (30 mL). The organic phase was dried over magnesium sulfate and evaporated to yield a colorless oil (400 mg) as a crude product.

The crude product was taken up in dichloromethane and purified via column chromatography over silica using a mixture of dichloromethane and methanol as the eluent. Subsequently, the corresponding fractions were collected and slowly concentrated on the rotary evaporator.

The purified product was dissolved in acetone. A colorless waxy fumarate salt (240 mg) was obtained after addition of fumaric acid.

### Example 5: Method of production of cyclopropyl(3-(2-(dimethylamino)ethyl)-1H-indol-1-yl)methanone

N,N-Dimethyltryptamine (1.0 mmol, 188 mg) was dissolved in tetrahydrofuran (16 mL) at 4° C. and was aerated with argon. A solution of n-butyl lithium in hexane (1.17 mmol, 0.47 mL at 2.5 M) was added dropwise through a septum. After addition of cyclopropane carbonyl chloride (1.5 mmol/0.13 mL) through the septum, the reaction mixture was stirred at 4° C. for an additional 60 minutes.

According to TLC and HPLC, complete consumption of the starting material has occurred.

The reaction mixture was evaporated, the resulting residue was dissolved in dichloromethane (50 mL) and extracted with water (50 mL) and saturated saline solution (50 mL). The organic phase was dried over magnesium sulfate and evaporated to yield a light yellowish oil as a crude product.

The crude product was dissolved in acetone. A colorless fumarate salt (225 mg) was obtained after addition of fumaric acid.

### Example 6: Method of production of cyclobutyl(3-(2-(dimethylamino)ethyl)-1H-indol-1-yl)methanone

N,N-Dimethyltryptamine (1.0 mmol, 188 mg) was dissolved in tetrahydrofuran (12 mL) at 4° C. and was aerated with argon. A solution of n-butyl lithium in hexane (1.17 mmol, 0.47 mL at 2.5 M) was added dropwise through a septum. After addition of cyclobutane carbonyl chloride (1.5 mmol/0.17 mL) through the septum, the reaction mixture was stirred at 4° C. for an additional 120 minutes.

According to TLC and HPLC, nearly complete consumption of the starting material has occurred.

The reaction mixture was evaporated, the resulting residue was dissolved in dichloromethane (70 mL) and extracted with water (30 mL) and saturated saline solution (30 mL). The organic phase was dried over magnesium sulfate and evaporated to yield a yellow oil as a crude product.

The crude product was dissolved in acetone. A colorless fumarate salt (186 mg) has obtained after addition of fumaric acid.

### Example 7: Method of production of 1-(3-(2-(dimethylamino)ethyl)-1H-indol-1-yl)-3-phenylproyan-1-one

N,N-Dimethyltryptamine (0.25 mmol, 47 mg) was dissolved in tetrahydrofuran (4 mL) at 4° C. and was aerated with argon. An n-butyl lithium in hexane solution (0.3 mmol, 0.12 mL at 2.5 M) was added dropwise through a septum. After addition of cyclopropane carbonyl chloride (0.38 mmol/0.06 mL) through the septum, the reaction mixture was stirred at 4° C. for an additional 80 minutes.

According to TLC and HPLC, complete consumption of the starting material has occurred.

The reaction mixture was evaporated, the resulting residue was dissolved in dichloromethane (30 mL) and extracted with water (30 mL) and saturated saline solution (30 mL). The organic phase was dried over magnesium sulfate.

The crude product was dissolved in acetone. A colorless fumarate salt (35 mg) has obtained after addition of fumaric acid.

### Example 8: Method of production of methyl 3-(2-(dimethylamino)ethyl)-1H-indole-1-carboxylate

N,N-Dimethyltryptamine (1.1 mmol, 200 mg) was dissolved in tetrahydrofuran (15 mL) at 4° C. and then was aerated with argon. An n-Butyl lithium solution in hexane (1.2 mmol, 0.48 mL, 2.5 M) was added dropwise through septum. After addition of methylchloroformate (1.7 mmol, 0.14 mL) through the septum, the reaction mixture was stirred at 4° C. for additional 30 min.

After removal of the ice bath, the reaction mixture was stirred at room temperature for another hour. According to TLC and HPLC, complete consumption of the starting material has occurred.

The reaction mixture was evaporated and the resulting residue dissolved in dichloromethane (50 mL) and extracted with water (50 mL) and saturated saline solution (50 mL). The organic phase was dried over magnesium sulfate and evaporated to yield a colorless oil as a crude product.

The crude product was taken up in dichloromethane and purified via column chromatography over silica using a mixture of dichloromethane and methanol as the eluent. Subsequently, the corresponding fractions were collected and slowly concentrated on the rotary evaporator to yield a colorless oil.

The purified product was dissolved in acetone. A colorless fumarate salt (95 mg) has obtained after addition of fumaric acid.

### Example 9: Method of production of ethyl 3-(2-(dimethylamino)ethyl)-1H-indole-1-carboxylate

N,N-dimethyltryptamine (1.33 mmol, 250 mg) was dissolved in chloroform (20 mL) at 25° C. 1,8-Diazabicyclo[5.4.0]undec-7-ene (1.33 mmol, 0.20 ml) and N,N-dimethylaminopyridine (2.66 mmol, 324 mg) were added and the flask was aerated with argon. After the addition of ethylchloroformate (13 mmol, 1.25 mL) through the septum, the reaction mixture was stirred at 25° C. for 48 hours.

The reaction mixture was diluted with dichloromethane (50 mL) and extracted with water (50 mL) and saturated saline solution (50 mL). The organic phase was dried over magnesium sulfate and evaporated to yield a light yellow oil as a crude product.

The crude product was taken up in 2 mL dichloromethane and purified via column chromatography over silica using a mixture of dichloromethane and methanol as the eluent. Subsequently, the corresponding fractions were collected and slowly concentrated on the rotary evaporator to yield a light yellow oil (95 mg).

### Example 10: Method of production of isopropyl 3-(2-(dimethylamino)ethyl)-1H-indole-1-carboxylate

N,N-dimethyltryptamine (1.33 mmol, 250 mg) was dissolved in chloroform (20 mL) at 25° C. 1,8-Diazabicyclo[5.4.0]undec-7-ene (6.6 mmol, 1.00 mL) and N,N-dimethylaminopyridine (2.66 mmol, 324 mg) were added and the flask was aerated with argon. After addition of isopropylchloroformate solution in toluene (4.7 mmol/4.7 mL) through the septum, the reaction mixture was stirred at 50° C. for 48 hours.

The reaction mixture was diluted with dichloromethane (50 mL) and extracted with water (50 mL) and saturated saline solution (50 mL). The organic phase was dried over magnesium sulfate and evaporated to yield a yellow oil (200 mg) as a crude product.

The crude product was taken up in 2 mL dichloromethane and purified via column chromatography over silica using a mixture of dichloromethane and methanol as the eluent. Subsequently, the corresponding fractions were collected and slowly concentrated on the rotary evaporator to yield a yellow oil (50 mg).

The purified product was dissolved in acetone. A brown fumarate salt (70 mg) was obtained after addition of fumaric acid.

### Example 11: Method of production of tert-butyl 3-(2-(dimethylamino)ethyl)-1H-indole-1-carboxylate

N,N-dimethyltryptamine (3.3 mmol, 620 mg) was dissolved in chloroform (35 mL) at 25° C. N,N-dimethylaminopyridine (9.0 mmol, 1.10 g) was added and the flask was aerated with argon. After addition of a solution of di-tert-butyl dicarbonate in tetrahydrofurane (3.3 mmol/3.1 mL) through the septum, the reaction mixture was stirred at 25° C. for 96 hours.

The reaction mixture was diluted with dichloromethane (180 mL) and extracted with water (130 mL) and saturated saline solution (130 mL). The organic phase was dried over magnesium sulfate and evaporated to yield a colorless oil as a crude product.

The crude product was taken up in dichloromethane and purified via column chromatography over silica using a mixture of dichloromethane and methanol as the eluent. Subsequently, the corresponding fractions were collected and slowly concentrated on the rotary evaporator to yield a yellow oil (310 mg).

The purified product was dissolved in acetone. A light yellow fumarate salt (205 mg) was obtained after addition of fumaric acid.

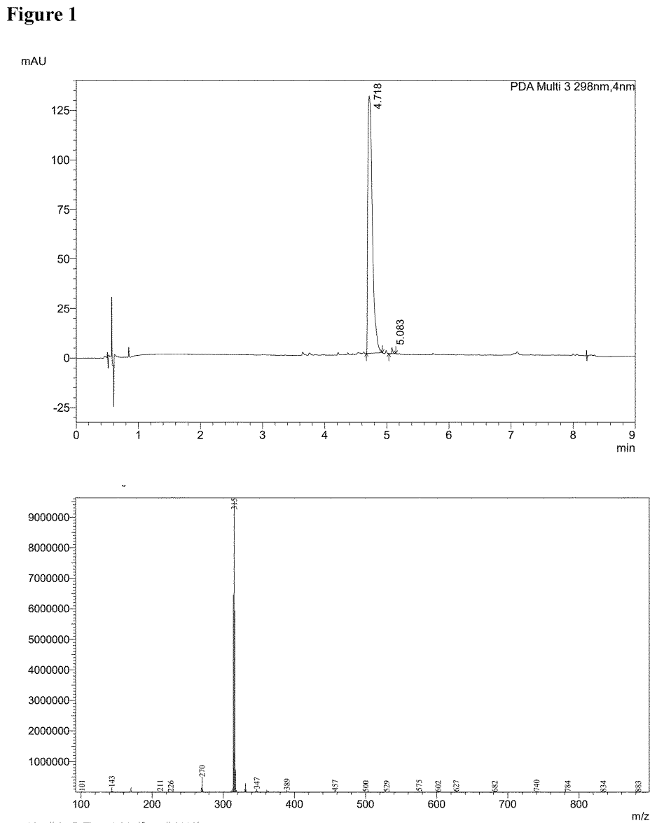

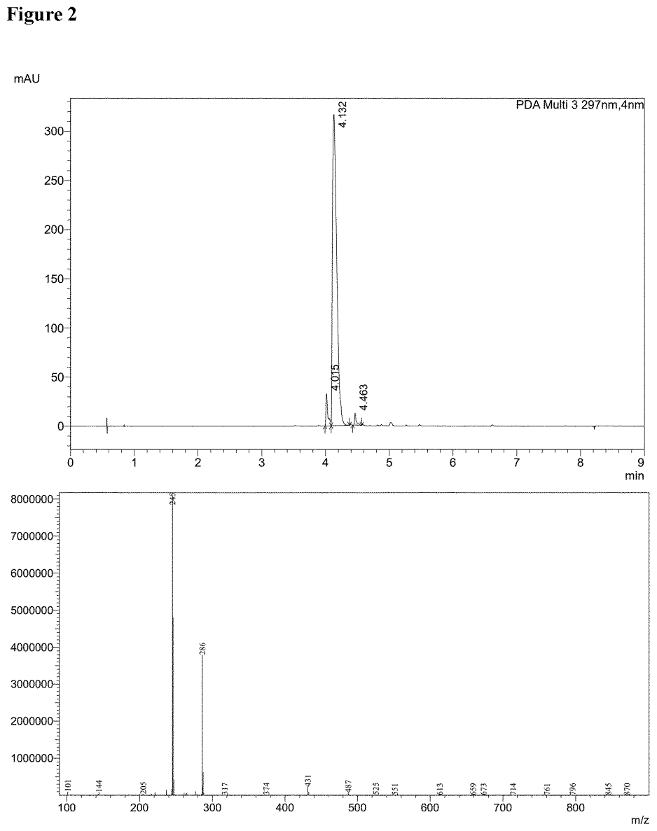

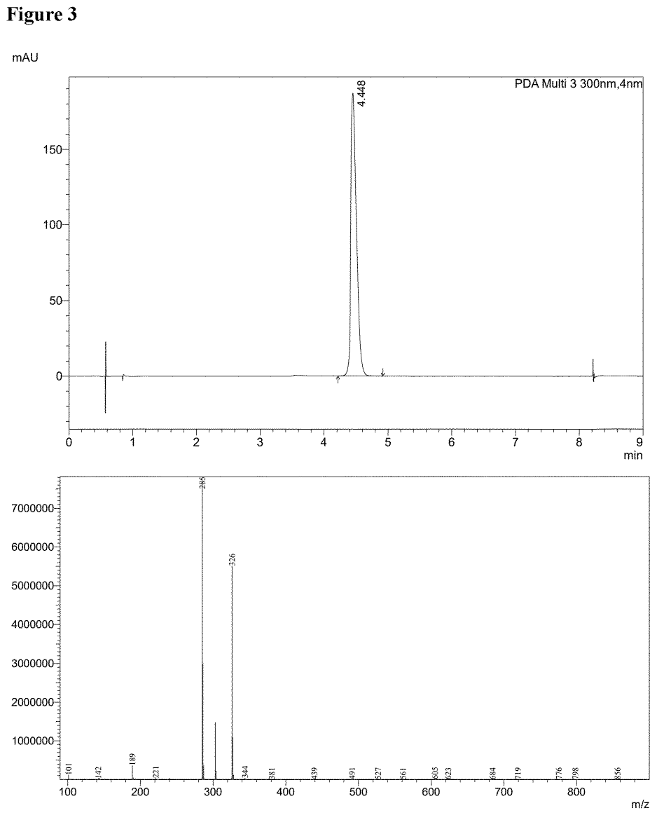

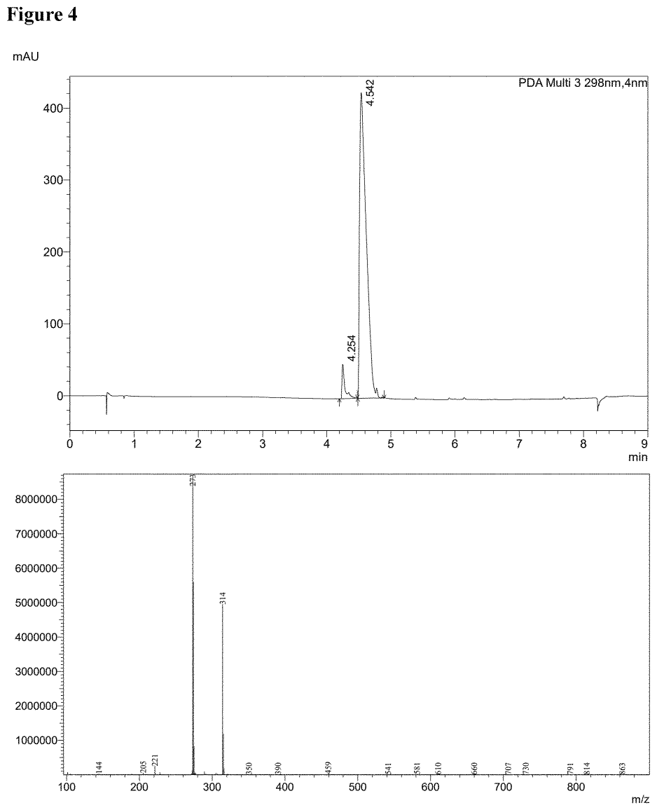

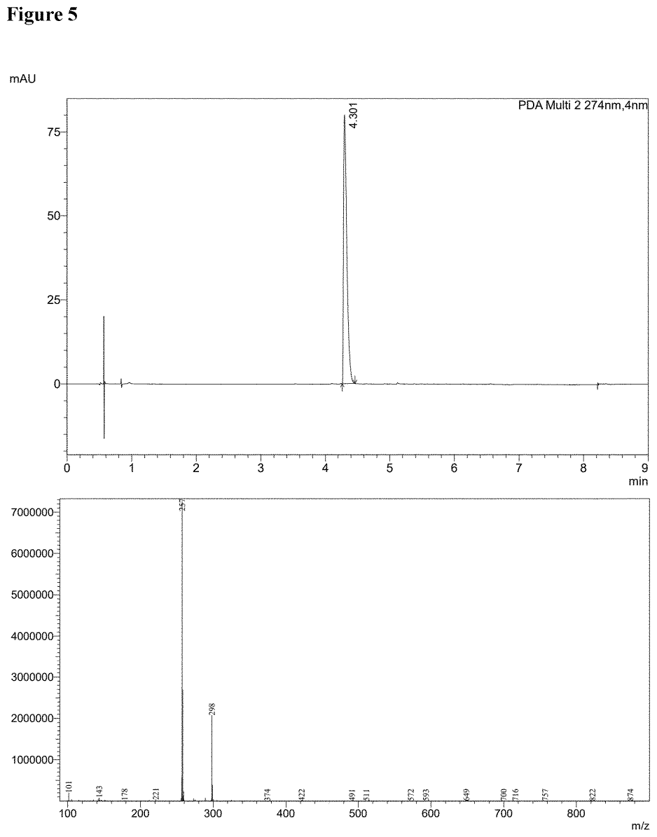

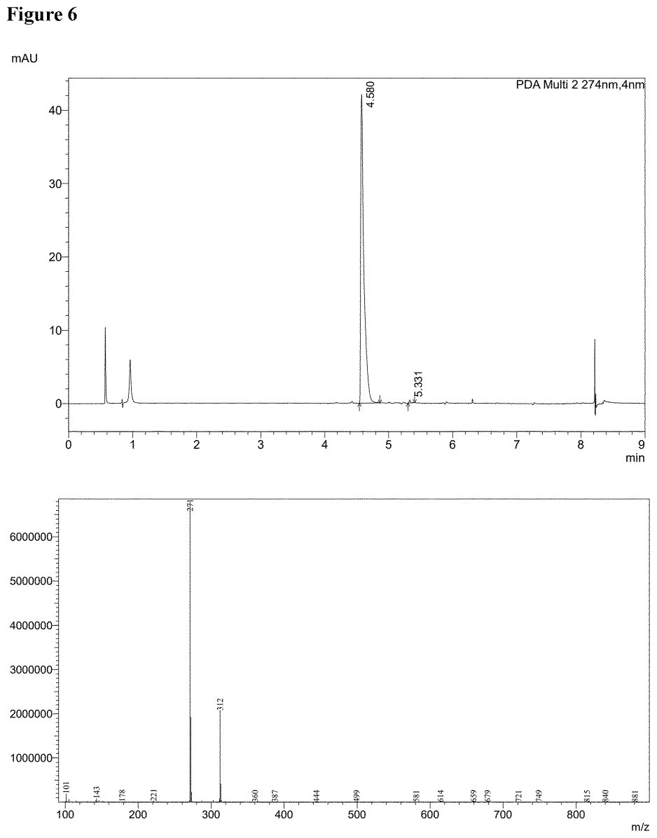

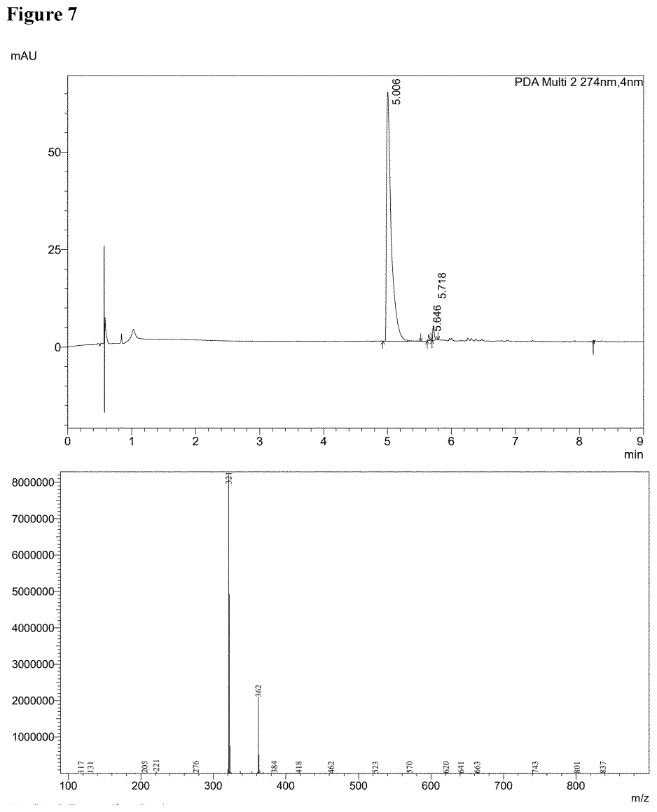

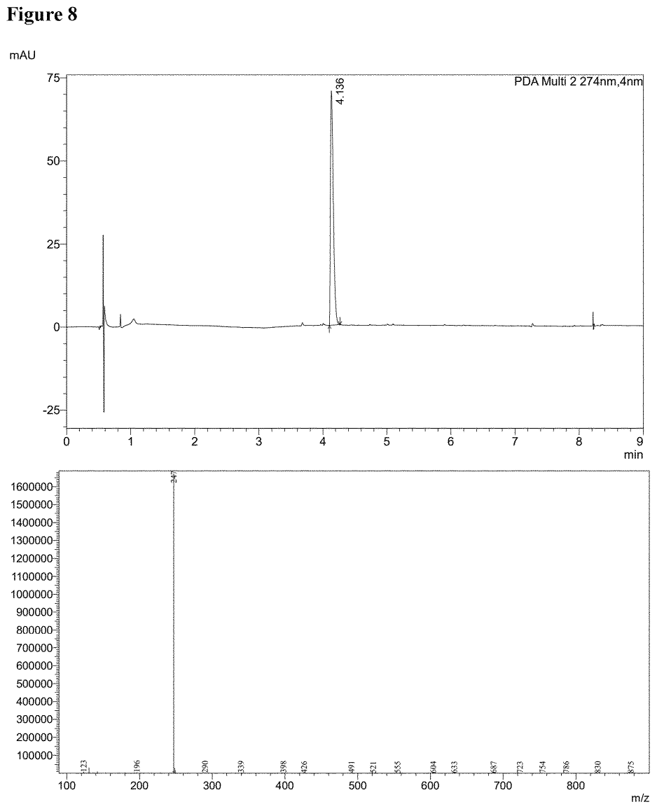

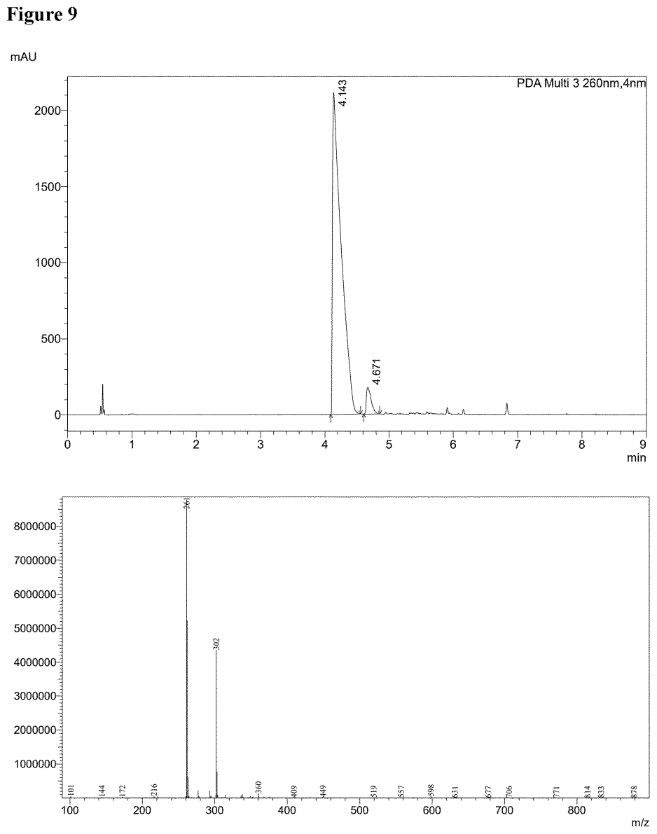

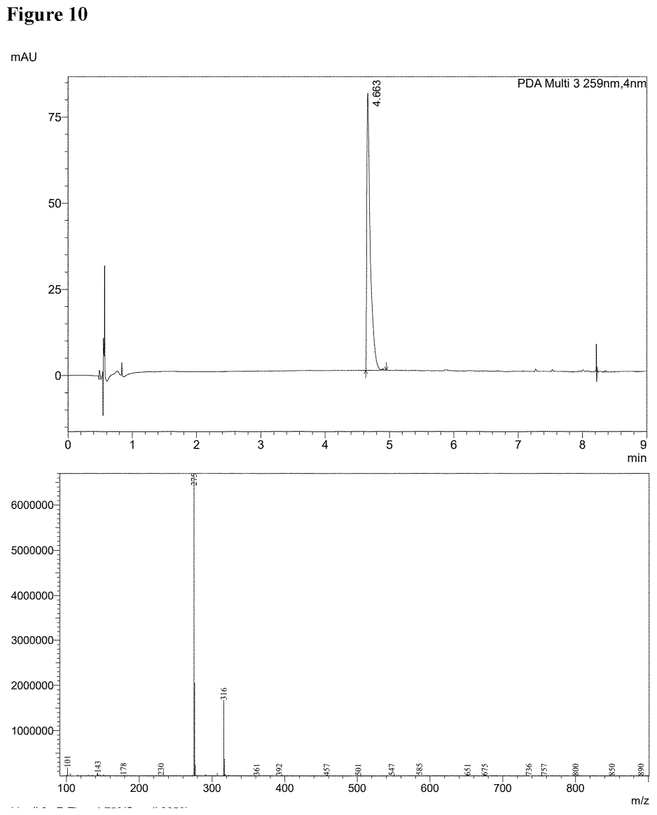

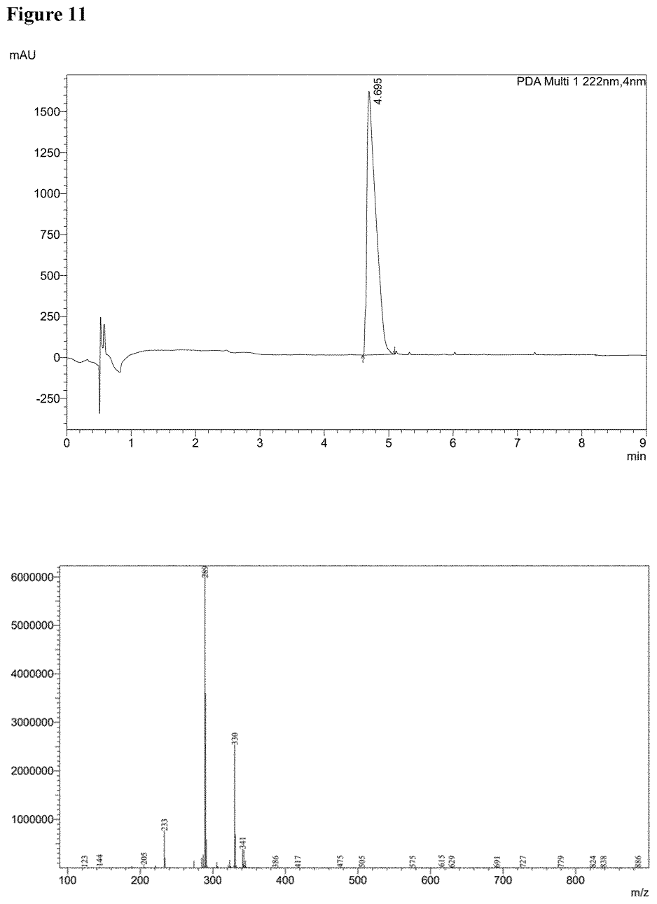

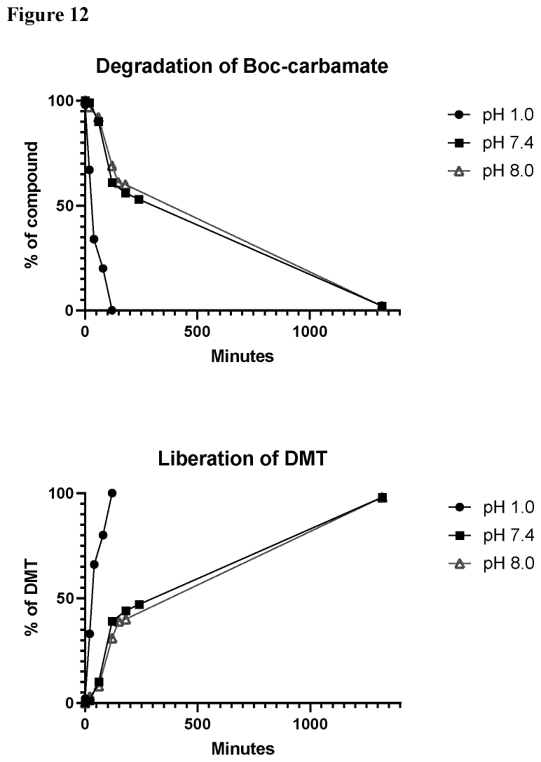

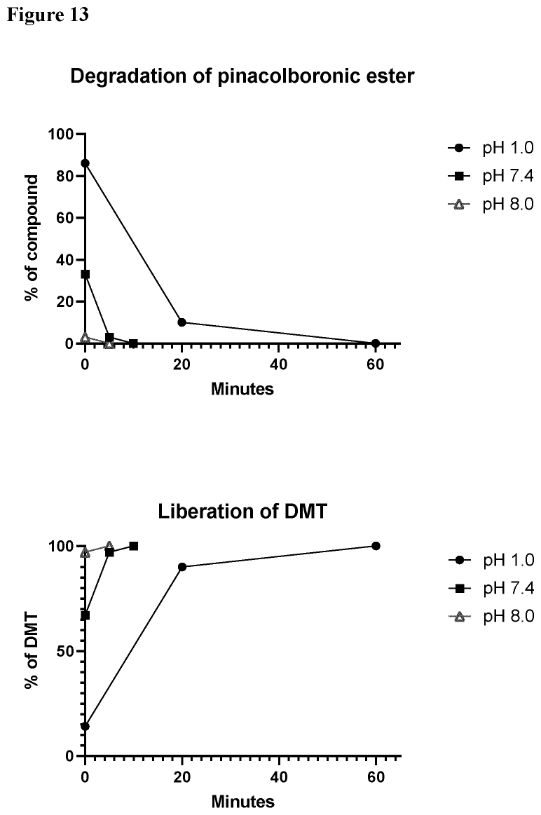

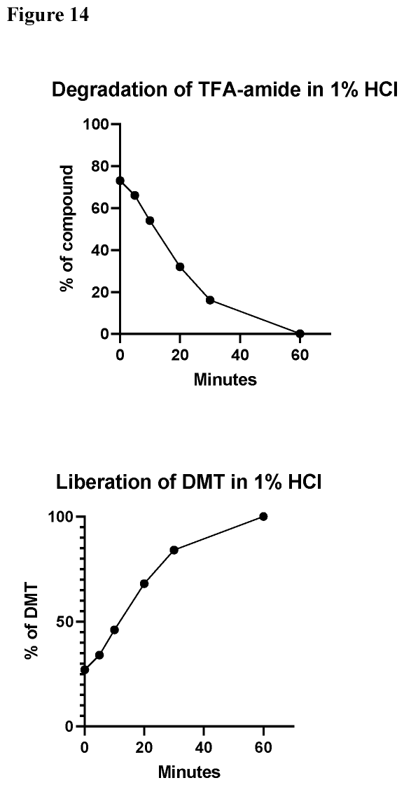

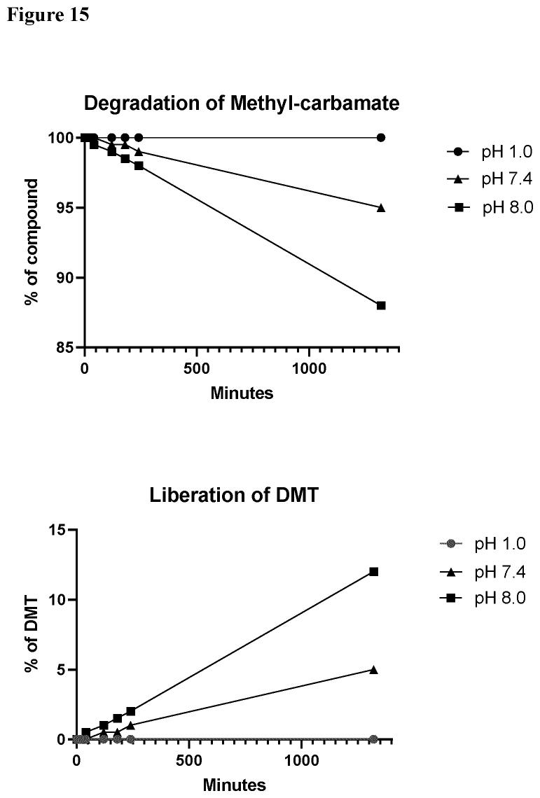

### Overview of the Compounds Produced in Examples 1 to 1.

| | | Method of |
|---|---|---|
| Com- | | production and |
| pound | | degradation/liberation |
| # | Structure and compound name | studies |
| 1 | 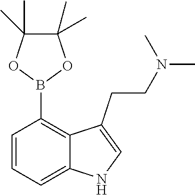 | Method of production according to Example 1, results presented in FIG. 1. Degradation studies and liberation of DMT at different pHs shown in FIG. 13. |
| | N,N-dimethyl-2-(4-(4,4,5,5-tetramethyl- | |
| | 1,3,2-dioxaborolan-2-yl)-1H-indol-3- | |
| | yl)ethan-1-amine | |
| 2 | 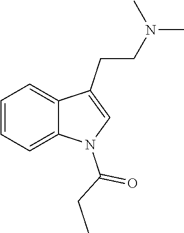 [SMILES: CCC(=O)n1cc(CCN(C)C)c2ccccc21] | Method of production according to Example 2, results presented in FIG. 2. |
| | 1-(3-(2-(dimethylamino)ethyl)-1H-indol-1- | |
| | yl)propan-1-one | |
| 3 | 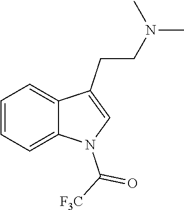 [SMILES: CN(C)CCc1cn(C(=O)C(F)(F)F)c2ccccc12] | Method of production according to Example 3, results presented in FIG. 3. Degradation studies and liberation of DMT shown in FIG. 14. |
| | 1-(3-(2-(dimethylamino)ethyl)-1H-indol-1- | |
| | yl)-2,2,2-trifluoroethan-1-one | |
| 4 | 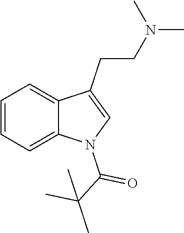 [SMILES: CN(C)CCc1cn(C(=O)C(C)(C)C)c2ccccc12] | Method of production according to Example 4, results presented in FIG. 4. |
| | 1-(3-(2-(dimethylamino)ethyl)-1H-indol-1- | |
| | yl)-2,2-dimethylpropan-1-one | |
| 5 | 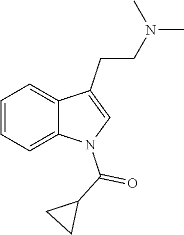 [SMILES: CN(C)CCc1cn(C(=O)C2CC2)c2ccccc12] | Method of production according to Example 5, results presented in FIG. 5. |
| | cyclopropyl(3-(2-(dimethylamino)ethyl)- | |
| | 1H-indol-1-yl)methanone | |
| 6 | 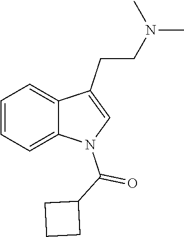 [SMILES: CN(C)CCc1cn(C(=O)C2CCC2)c2ccccc12] | Method of production according to Example 6, results presented in FIG. 6. |
| | cyclobutyl(3-(2-(dimethylamino)ethyl)-1H- | |
| | indol-1-yl)methanone | |
| 7 | 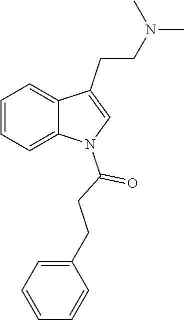 [SMILES: CN(C)CCc1cn(C(=O)CCc2ccccc2)c2ccccc12] | Method of production according to Example 7, results presented in FIG. 7. |
| | 1-(3-(2-(dimethylamino)ethyl)-1H-indol-1- | |
| | yl)-3-phenylpropan-1-one | |
| 8 | 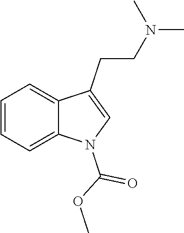 [SMILES: COC(=O)n1cc(CCN(C)C)c2ccccc21] | Method of production according to Example 8, results presented in FIG. 8. Degradation studies and liberation of DMT at different pHs shown in FIG. 15. |
| | methyl 3-(2-(dimethylamino)ethyl)-1H- | |
| | indole-1-carboxylate | |
| 9 | 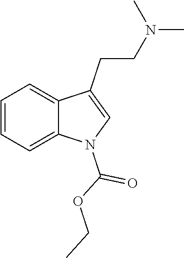 [SMILES: CCOC(=O)n1cc(CCN(C)C)c2ccccc21] | Method of production according to Example 9, results presented in FIG. 9. |
| | ethyl 3-(2-(dimethylamino)ethyl)-1H- | |
| | indole-1-carboxylate | |
| 10 | 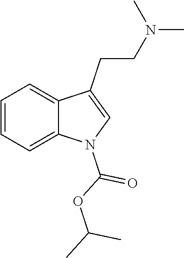 [SMILES: CC(C)OC(=O)n1cc(CCN(C)C)c2ccccc21] | Method of production according to Example 10, results presented in FIG. 10. |
| | isopropyl 3-(2-(dimethylamino)ethyl)-1H- | |
| | indole-1-carboxylate | |
| 11 | 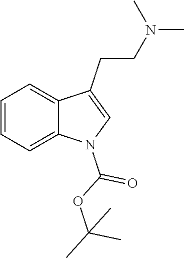 [SMILES: CN(C)CCc1cn(C(=O)OC(C)(C)C)c2ccccc12] | Method of production according to Example 11, results presented in FIG. 11. Degradation studies and liberation of DMT at different pHs shown in FIG. 12. |
| | tert-butyl 3-(2-(dimethylamino)ethyl)-1H- | |
| | indole-1-carboxylate | |

### Example 12: Stability Studies of Novel DMT Prodrugs in HCl and Phosphate Buffers

Four compounds (i.e., methyl carbamate [compound 8, Example 8], boc carbamate [compound 11, Example 3], pinacol boronic ester [compound 1, Example 1] and TFA-amide [compound 3, Example 3]) were tested for their stability in 1% hydrochloric acid (HCl) as well as in phosphate buffers (0.1 M) at pH 7.4 and pH 8.0, respectively. These conditions were selected to assess the chemical stability of these compounds at a pH corresponding to different physiological conditions. Dilute hydrochloric acid (pH 1) was used to mimic the low pH of gastric acid within the stomach, while potassium phosphate buffers of pH 7.4 and pH 8.0 were used to mimic conditions in human blood and small intestine, respectively.

### Methods

4 mg of each test compound was diluted in 4 mL of either a 1% HCl solution or the respective phosphate buffer (0.1 M), resulting in a solution of 1 mg/mL each. Regarding the acidic testing with hydrochloric acid, 2 mL of aqueous test compound solutions were then added to 2 mL of 2% (v/v) in distilled water, yielding a final HCl concentration of 0.32 mM (pH 0.5). Test solutions were incubated at 37° C. with constant stirring for approximately 21 hours. The concentrations of the parent compound were analyzed at different timepoints using LC/MS. The concentrations of the parent prodrug and released drug were expressed relative to the starting concentration of the parent prodrug.

### Results

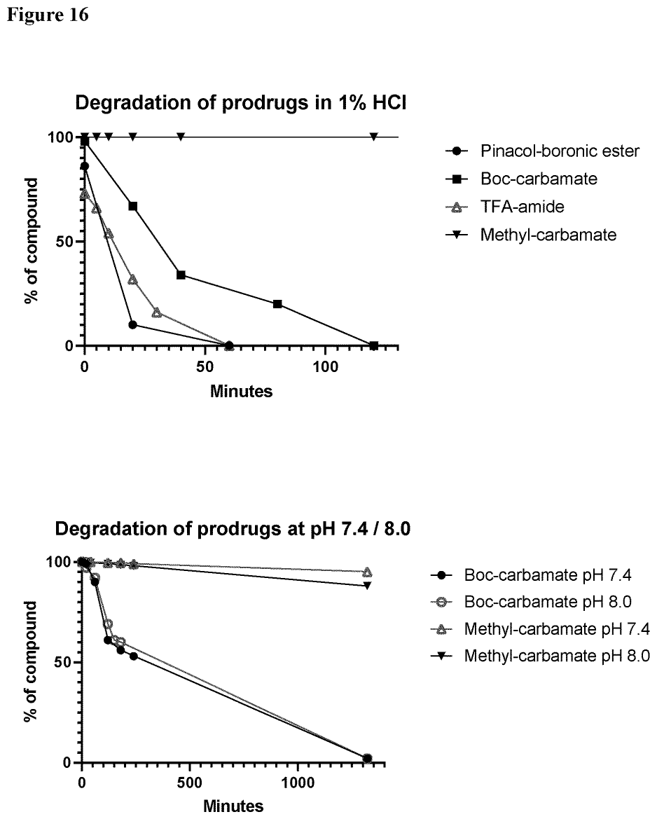

The results obtained in this experiment are shown in FIGS. 12-16. Degradation experiments using compound 3 at pH 7.4 and pH 8.0, respectively, showed essentially instantaneous degradation (100%) of the prodrug concomitant with liberation of DMT. No liberation of DMT was observed upon degradation of compound 8 when the experiment was performed in a HCl solution at pH 1.0.

### Conclusions

The results show that all tested prodrugs degrade under either acidic, neutral or basic conditions and reliably release the intended drug DMT. Furthermore, both the TFA-amide (compound 3) as well as the pinacol boronic ester (compound 1) are capable of releasing DMT rapidly within minutes. Both prodrugs tend to exhibit their maximum stability under acidic conditions, making them favorable for oral ingestion. Boc carbamate (compound 11) proves to be significantly more stable under all tested conditions. Conversion to DMT proceeded efficiently throughout the entire experiment at all pHs. Under acidic conditions, a remarkably fast release time of two hours was demonstrated. Compared to the other tested prodrugs, methyl carbamate (compound 8) presents complementary characteristics. No conversion was observed under acidic conditions. At both pH 7.4 and pH 8.0, a continuous release of DMT was observed throughout the experiment.

Consequently, the compounds show both immediate and slow but significant degradation to DMT, depending on their mode of chemical derivatization. These data support the conclusion that the compounds of the present invention are specifically designed to release the drug at characteristic pH values or at distinct conversion rates and can therefore be used as a medicament.

### Example 13: Method of production of 2-(1-(cyclopropylsulfonyl)-1H-indol-3-yl)-N,N-dimethylethan-1-amine

N,N-Dimethyltryptamine (8 mmol/1.5 g) was dissolved in tetrahydrofuran (45 ml) at 4° C. and then was aerated with argon. N-Butyl lithium solution in hexane (1.2 mmol/3.8 ml 2.5 M) added dropwise through septum. After the addition of cyclopropanesulfonyl chloride (12 mmol/1.3 ml) diluted in 5 ml THE through the septum, the reaction mixture was stirred at 4° C. for additional 30 min.

After removal of the ice bath, the reaction mixture was stirred over night at room temperature. According to HPLC, 55% conversion of starting material has occurred.

The reaction mixture was diluted with dichloromethane (80 ml) and extracted with water (80 ml) and saturated saline solution (80 ml). The organic phase was dried over magnesium sulfate and evaporated to yield 2.0 g of colorless oil as a crude product.

The raw product was taken up in dichloromethane and purified via column chromatography over silica using dichloromethane/ethanol as the eluent. Subsequently, the corresponding fractions were collected and slowly concentrated on the rotary evaporator to yield 1.6 g colorless wax (yield 70%).

### Example 14: Method of production of 2-(1-(ethylsulfonyl)-1H-indol-3-yl)-N,N-dimethylethan-1-amine

N,N-Dimethyltryptamine (5.3 mmol/1.0 g) was dissolved in tetrahydrofuran (30 ml) at 4° C. and then was aerated with argon. N-Butyl lithium solution in hexane (3.6 mmol/1.44 ml 2.5 M) added dropwise through septum. After the addition of ethanesulfonyl chloride (4.5 mmol/0.35 ml) diluted in 5 ml THE through the septum, the reaction mixture was stirred at 4° C. for additional 30 min.

After removal of the ice bath, the reaction mixture was stirred over night at room temperature. According to HPLC, 70% conversion of starting material has occurred.

The reaction mixture was diluted with dichloromethane (80 ml) and extracted with water (80 ml) and saturated saline solution (80 ml). The organic phase was dried over magnesium sulfate and evaporated to yield 1.8 g of colorless oil as a crude product.

The raw product was taken up in dichloromethane and purified via column chromatography over silica using dichloromethane/ethanol as the eluent. Subsequently, the corresponding fractions were collected and slowly concentrated on the rotary evaporator to yield 0.82 g colorless wax.

The purified product was dissolved in ethanol. A colorless fumarate salt 1.05 g has obtained by addition of fumaric acid (yield 55%).

### Example 15: Method of production of 2-(1-(methylsulfonyl)-1H-indol-3-yl)-N,N-dimethylethan-1-amine

N,N-Dimethyltryptamine (3 mmol/0.56 g) was dissolved in tetrahydrofuran (25 ml) at 4° C. and then was aerated with argon. N-Butyl lithium solution in hexane (1.2 mmol/3.8 ml 2.5 M) added dropwise through septum. After the addition of cyclopropanesulfonyl chloride (12 mmol/1.3 ml) diluted in 5 ml THE through the septum, the reaction mixture was stirred at 4° C. for additional 30 min.

After removal of the ice bath, the reaction mixture was stirred over night at room temperature. According to HPLC, 55% conversion of starting material has occurred.

The reaction mixture was diluted with dichloromethane (80 ml) and extracted with water (80 ml) and saturated saline solution (80 ml). The aqueous phase was extracted with ethyl acetate and tetrahydrofuran. The combined organic phase was dried over magnesium sulfate and evaporated to yield 0.81 g of colorless solid as a crude product.

The raw product was taken up in dichloromethane and purified via column chromatography over silica using dichloromethane/ethanol as the eluent. Subsequently, the corresponding fractions were collected and slowly concentrated on the rotary evaporator to yield 0.66 g colorless solid.

### Example 16: Interaction of Selected Compounds with the Human Serotonin Transporter

The goal of the investigation was to ascertain that (i) the compounds interact with the respective targets as described above, with or without pre-treatment with hydrochloric acid, mimicking the gastric passage, and (ii) that the compounds trigger transporter-mediated efflux as known from the literature (including our own). Dimethyltryptamine (DMT) was as comparator/control substance for the experiments.

### Cell Culture

Human embryonic kidney (HEK) 293 cells stably expressing human SERT were maintained in humidified atmosphere (37° C., 5% CO₂) in Dulbecco's Modified Eagle Medium (DMEM), supplemented with 10% heat-inactivated fetal calf serum (FCS), streptomycin (100 μg×100 mL⁻¹) and penicillin (100 U×100 mL⁻¹). Geneticin (50 μg×mL⁻¹) was used as selection antibiotic.

### Uptake Inhibition Experiments

One day before the experiment, HEK293-SERT cells were seeded onto poly-D-lysine coated 96 well plates at a density of 30,000 cells per well in a final volume of 200 μL. For uptake experiments, the cell culture medium was replaced with 300 μL Krebs-HEPES buffer (KHB; 25 mM HEPES, 120 mM NaCl, 5 mM KCl, 1.2 mM CaCl₂, 1.2 mM MgSO₄, and 5 mM D-glucose, pH 7.3). Then, the buffer was replaced with various concentrations of test compounds for 5 minutes; subsequent to that, 0.1 μM [³H]5-HT in KHB was added to yield a total volume of 50 μL in the well. For determination of non-specific uptake, paroxetine was used (3 μM).

Uptake was terminated after 60 s by washing the cells with 200 μL ice-cold KHB. Subsequently, the cells were lysed with 200 μL Ultima Gold™ XR liquid scintillation cocktail, and the amount of tritium in the cells was measured with a Wallac 1450 MicroBeta® TriLux liquid scintillation counter. Monoamine uptake data were fitted by nonlinear regression, Vmax and Km values were calculated from Michaelis Menten's least-squares fit with GraphPad Prism (Prism 9.0.2, GraphPad Software, San Diego, CA, USA).

### Release Experiments

The day prior to the assay, stably expressing HEK293-SERT cells were seeded in a perfusion chamber of an IBIDI microfluidic chip (μ-Slide VI 0.5 Glass Bottom, ibidi GmbH). Chambers were previously coated with poly-D-lysine for 20 min at 37° C. and then cells were seeded at a density of approximately 1-10⁶ cells×mL⁻¹.

For the release experiment, cells were loaded with radioactive substrate 0.08 M [³H]5-HT for 20 min at 37° C. and then perfused with different solutions at constant flow rate (0.6 μL min⁻¹). The assay included washout with KHB (pH 7.3; 20 min) to establish a stable baseline efflux, perfusion of cells with KHB (6 min) and collection of the baseline samples, injection of control compounds (para-chloroamphetamine 3 μM) or test compounds at various concentrations for 10 min. The cells were flushed using sodium dodecyl sulfate (SDS) to retrieve the residual radioactivity (6 min). Interaction of detergent with the cells lysed the cell membranes and all the radioactive substrate was collected at the outlet.

Radioactivity was quantified with a beta-scintillation counter (PerkinElmer, Waltham, MA, USA). The released amount of tritiated substrate for each fraction was expressed as the amount of released tritium compared to the amount remaining in the cell at the end of the previous fraction.

### Test Compounds

We tested Example 1, Example 4, Example 13 in both uptake inhibition and release assays, and Example 15 in uptake inhibition assays. Drugs were tested either as the undecomposed prodrug or after treatment with 0.5% HCl at 37° C. for 24 hours in order to simulate digestion of the prodrug by gastric acid inside the body after peroral administration. DMT was tested dissolved only in DMSO.

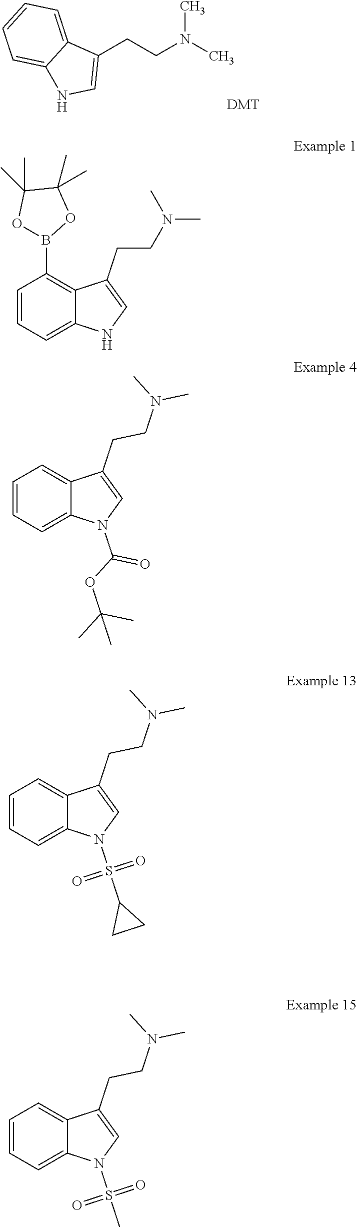

### Results

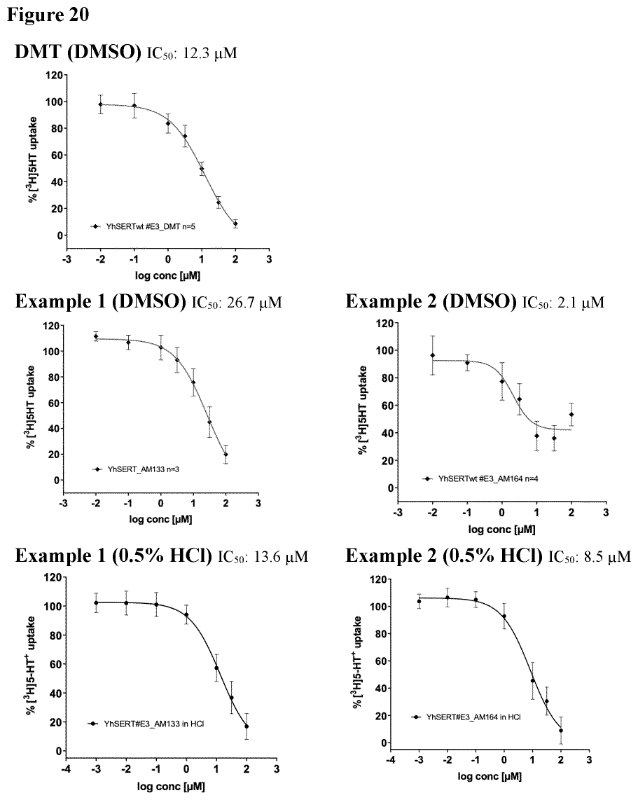

Uptake inhibition assays at SERT: In these uptake inhibition assays, all compounds showed interaction with SERT, i.e. the compounds inhibited substrate-uptake in a competitive manner. However, the dissolution of the test compounds was much better in the HCl condition compared to the DMSO-dissolution. Under HCl, the dissolved compounds of Example 1 and Example 4 reached very close IC₅₀ values compared to DMT. The results are shown in FIG. 20.

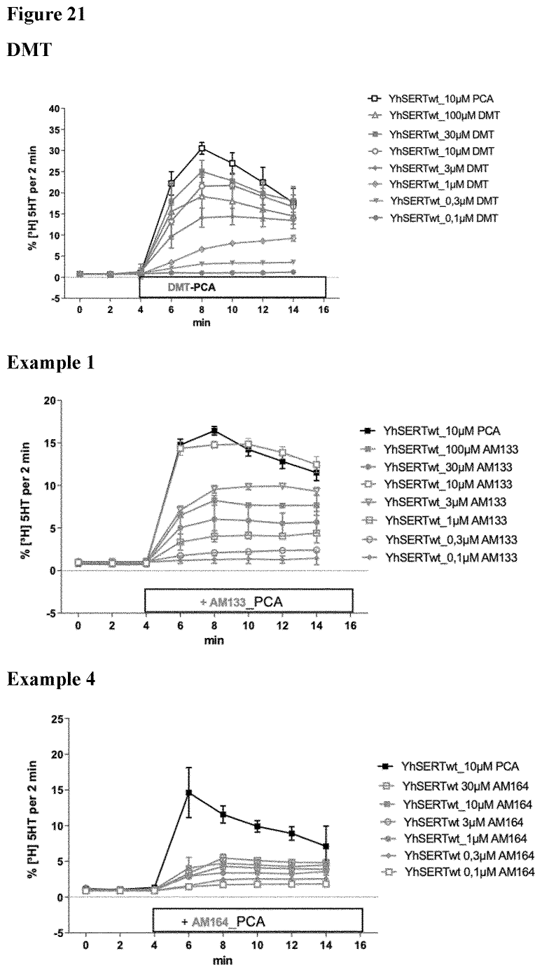

Release assays: The release assays show unanimously that the compound DMT induces efflux in a manner comparable to pCA (full releaser) or MDMA, with almost full efficacious release being observable in HEK293-SERT cells. Example 1 (dissolved in HCl) does not elicit full release, compared to DMT, however, it certainly induces efflux via SERT; Example 4 (dissolved in HCl) induces even less efflux, compared to pCA and also Example 1. The results are shown in FIG. 21.

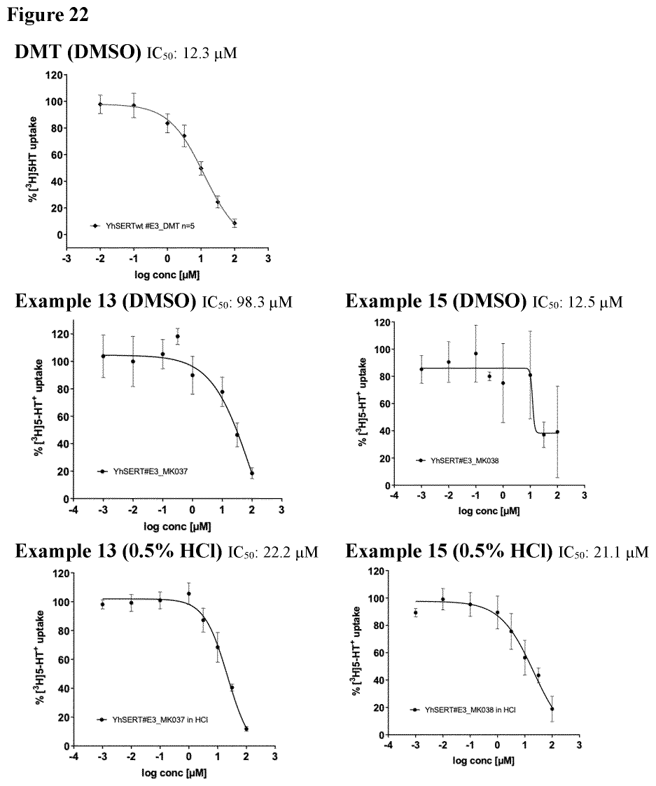

Uptake inhibition assays: We observed that with the substances of Example 13 and Example 15, dissolution in DMSO was not very well achieved, only in HCl, both compounds dissolved and reached activity, however, to a much lower grade compared to DMT and also Examples 1 and 4. Nevertheless, both compounds interacted with SERT, albeit to a lesser extent compared to Examples 1 and 4. The results are shown in FIG. 22.

TABLE
Uptake inhibition, IC₅₀ values

| | SERT | |
|---|---|---|
| DMT (DMSO) | 12.3 | μM |
| Example 1 (DMSO) | 26.7 | μM |
| Example 4 (DMSO) | ~2.1 | μM |
| Example 1 (HCl) | 13.6 | μM |
| Example 4 (HCl) | 8.3 | μM |
| Example 13 (DMSO) | 93.8 | μM |
| Example 15 (DMSO) | ~12.5 | μM |
| Example 13 (HCl) | 22.2 | μM |
| Example 15 (HCl) | 21.1 | μM |

### Conclusion

In conclusion, due to their similarity to DMT, the compounds of Example 1 and Example 4 were tested with or without pre-treatment conditions. Under all conditions, the compounds were found to inhibit the serotonin transporters unanimously, however, in DMSO conditions, the potency to interact with SERT is considerably weaker. This means that both compounds exert an activity in the absence of HCl-treatment but elicit much better efficacy when this treatment has been applied. In addition, both compounds were tested whether they can also induce transporter-mediated efflux. Indeed, in comparison to DMT, Example 1 is capable of inducing transporter-mediated efflux and also elicits full release similar to pCA. Example 4, however, yields a lower efflux compared to both DMT and Example 1, most likely owing to the fact that it does not dissociate as efficiently.

When comparing the results to the literature, uptake inhibition in human platelets expressing SERT by DMT was around 10 μM, which is a pretty similar value compared to our HEK293-SERT cells.

Example 13 and Example 15 were tested in uptake inhibition assays with or without pre-treatment conditions: Without pre-treatment, interaction with the transporter was scarce. However, after treatment with HCl, which was done in order to simulate the gastric passage, uptake inhibition was seen in a range lower than the one seen with DMT, Example 1 and Example 4, however, the IC₅₀-values are higher by a factor of about 2.

1. A compound according to the general formula (I), or a pharmaceutically acceptable salt thereof 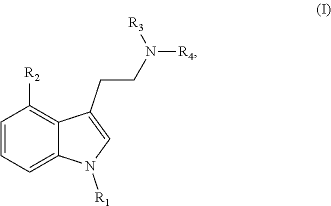
wherein:
R₁ is selected from the group consisting of hydrogen, 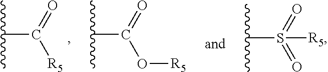
wherein R₅ is selected from C₁₋₁₂ alkyl, C₂₋₁₂ alkenyl, C₂₋₁₂ alkynyl, —CH₂—O—(CH₂CH₂O)ₘ—R₆, —O—(CH₂CH₂O)ₙ—R₆, —NH(CH₂CH₂O)ₚ—R₆, phenyl, benzyl, phenethyl, pyridyl, cycloalkyl, (cycloalkyl)alkyl, cycloalkenyl, (cycloalkenyl)alkyl, 5- or 6-membered heterocyclyl, (5- or 6-membered heterocyclyl)alkyl, and 9-fluorenylmethyl, wherein each one of the aforementioned groups is optionally substituted;
m, n and p are each independently an integer from 1 to 5;
R₆ is hydrogen or is selected from C₁₋₆ alkyl, aryl, heteroaryl, cycloalkyl, and heterocyclyl, wherein each one of the aforementioned groups is optionally substituted;
R₂ is selected from hydrogen and 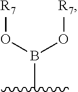
wherein each R₇ is independently selected from hydrogen and R₈, or wherein the two groups R₇, together with the —O—B—O— moiety that they are attached to, form a heterocyclyl which is optionally substituted;
R₈ is selected from C₁₋₁₂ alkyl, C₂₋₁₂ alkenyl, C₂₋₁₂ alkynyl, —CH₂—O—(CH₂CH₂O)ₘ—R₉, —O—(CH₂CH₂O)ₙ—R₉, —NH(CH₂CH₂O)ₚ—R₉, phenyl, benzyl, phenethyl, pyridyl, cycloalkyl, (cycloalkyl)alkyl, cycloalkenyl, (cycloalkenyl)alkyl, 5- or 6-membered heterocyclyl, (5- or 6-membered heterocyclyl)alkyl, and 9-fluorenylmethyl, wherein each one of the aforementioned groups is optionally substituted;
m, n and p are as defined above;
R₉ is hydrogen or is selected from C₁₋₆ alkyl, aryl, heteroaryl, cycloalkyl, and heterocyclyl, wherein each one of the aforementioned groups is optionally substituted;
R₃ is selected from hydrogen, methyl and ethyl;
R₄ is selected from hydrogen, methyl and ethyl; and
wherein at least one of R₁ and R₂ is not hydrogen.

2. The compound of claim 1, wherein both R₁ and R₂ are not hydrogen.

3. The compound according to claim 1 or 2, wherein R₂ is

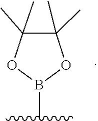

4. The compound according to any one of claims 1 to 3, wherein R₁ is

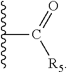

5. The compound according to any one of claims 1 to 4, wherein R₅ is optionally substituted C₁₋₄ alkyl.

6. The compound according to any one of claims 1 to 4, wherein R₅ is optionally substituted cycloalkyl.

7. The compound according to any one of claims 1 to 4, wherein R₅ is optionally substituted phenethyl.

8. The compound according to any one of claims 1 to 3, wherein R₁ is

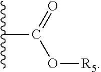

9. The compound according to claim 8, wherein R₅ is optionally substituted C₁₋₄ alkyl.

10. The compound according to any one of claims 1 to 3, wherein R₁ is selected from

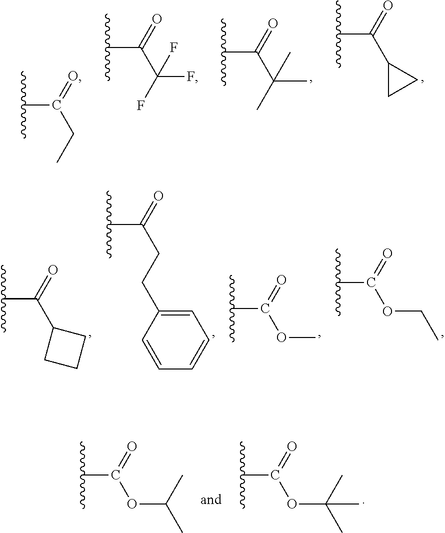

11. The compound according to any one of claims 1 to 3, wherein R₁ is

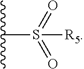

12. The compound according to claim 11, wherein R₅ is optionally substituted C₁₋₄ alkyl, preferably methyl.

13. The compound according to claim 11, wherein R₅ is optionally substituted cycloalkyl, preferably cyclopropyl.

14. The compound according to any one of claims 1 to 13, wherein both R₃ and R₄ are methyl.

15. The compound according to claim 1, wherein the compound is selected from the group consisting of:
N,N-dimethyl-2-(4-(4,4,5,5-tetramethyl-1,3,2-dioxaborolan-2-yl)-1H-indol-3-yl)ethan-1-amine,
1-(3-(2-(dimethylamino)ethyl)-1H-indol-1-yl)propan-1-one,
1-(3-(2-(dimethylamino)ethyl)-1H-indol-1-yl)-2,2,2-trifluoroethan-1-one,
1-(3-(2-(dimethylamino)ethyl)-1H-indol-1-yl)-2,2-dimethylpropan-1-one,
cyclopropyl(3-(2-(dimethylamino)ethyl)-1H-indol-1-yl)methanone,
cyclobutyl(3-(2-(dimethylamino)ethyl)-1H-indol-1-yl)methanone,
1-(3-(2-(dimethylamino)ethyl)-1H-indol-1-yl)-3-phenylpropan-1-one,
methyl 3-(2-(dimethylamino)ethyl)-1H-indole-1-carboxylate,
ethyl 3-(2-(dimethylamino)ethyl)-1H-indole-1-carboxylate,
isopropyl 3-(2-(dimethylamino)ethyl)-1H-indole-1-carboxylate,
tert-butyl 3-(2-(dimethylamino)ethyl)-1H-indole-1-carboxylate,
2-(1-(cyclopropylsulfonyl)-1H-indol-3-yl)-N,N-dimethylethan-1-amine,
2-(1-(ethylsulfonyl)-1H-indol-3-yl)-N,N-dimethylethan-1-amine, and
2-(1-(methylsulfonyl)-1H-indol-3-yl)-N,N-dimethylethan-1-amine;
or a pharmaceutically acceptable salt thereof.

16. A pharmaceutical composition comprising a compound of any one of claims 1 to 15 and optionally one or more pharmaceutically acceptable excipient(s) and/or carrier(s).

17. The compound of any one of claims 1 to 15 or the pharmaceutical composition of claim 16 for use as a medicament.

18. The compound of any one of claims 1 to 15 or the pharmaceutical composition of claim 16 for use in the treatment, amelioration or prevention of a serotonin 5-HT_{2A} receptor associated disease.

19. The compound of any one of claims 1 to 15 or the pharmaceutical composition of claim 16 for use in the treatment, amelioration or prevention of an anxiety disorder, attention deficit hyperactivity disorder (ADHD), depression, cluster headache, diminished drive, burn-out, bore-out, migraine, Parkinson's disease, pulmonary hypertension, schizophrenia, an eating disorder, nausea, vomiting, alcohol/drug dependency, or post-traumatic stress disorder (PTSD).

20. Use of the compound of any one of claims 1 to 15 for the preparation of a medicament for the treatment, amelioration or prevention of a serotonin 5-HT_{2A} receptor associated disease.

21. Use of the compound of any one of claims 1 to 15 for the preparation of a medicament for the treatment, amelioration or prevention of an anxiety disorder, attention deficit hyperactivity disorder (ADHD), depression, cluster headache, diminished drive, burn-out, bore-out, migraine, Parkinson's disease, pulmonary hypertension, schizophrenia, an eating disorder, nausea, vomiting, alcohol/drug dependency, or post-traumatic stress disorder (PTSD).

22. A method of treating, ameliorating or preventing a serotonin 5-HT_{2A} receptor associated disease, the method comprising administering a therapeutically effective amount of the compound of any one of claims 1 to 15 to a subject in need thereof.

23. A method of treating, ameliorating or preventing an anxiety disorder, attention deficit hyperactivity disorder (ADHD), depression, cluster headache, diminished drive, burn-out, bore-out, migraine, Parkinson's disease, pulmonary hypertension, schizophrenia, an eating disorder, nausea, vomiting, alcohol/drug dependency, or post-traumatic stress disorder (PTSD), the method comprising administering a therapeutically effective amount of the compound of any one of claims 1 to 15 to a subject in need thereof.
